(19) United States
(12) Patent Application Publication
(10) Pub. No.: US 2006/0047118 A1
(43) Pub. Date: Mar. 2, 2006

(54) New pteridinones as PLK inhibitors

(75) Inventors: Heinz Stadtmueller, Gaweinstal (AT); Harald Engelhardt, Ebreichsdorf (AT); Andreas Schoop, Vienna (AT); Martin Steegmaier, Vienna (AT)

Correspondence Address: MICHAEL P. MORRIS;BOEHRINGER INGELHEIM CORPORATION, 900 RIDGEBURY RD, P. O. BOX 368, RIDGEFIELD, CT, 06877-0368, US

(73) Assignee: Boehringer Ingelheim International GmbH, Ingelheim (DE)

(21) Appl. No.: 11/211,048

(22) Filed: Aug. 24, 2005

(30) Foreign Application Priority Data
Aug. 26, 2004 (EP) EP 04 020 291

(51) Int. Cl. C07D 487/02; C07D 475/02

(52) U.S. Cl. 544/184; 544/279; 544/257

(57) ABSTRACT

Disclosed compounds of general formula (1) 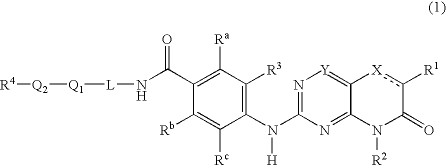 wherein
L, Q₁, Q₂, X, Y, R^{a}, R^{b}, R^{c}, R¹, R², R³ and R⁴ are defined as in claim 1, which are suitable for the treatment of diseases characterised by excessive or abnormal cell proliferation, and the use thereof for preparing a pharmaceutical composition with the above-mentioned properties.

### APPLICATION DATA

This application claims benefit to European Patent Application no. EP 04 020 291.3 filed Aug. 26, 2004.

The present invention relates to new pteridinones of general formula (1) 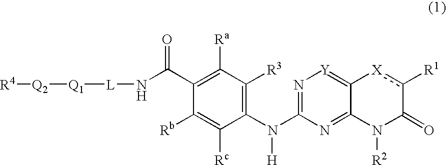
while the groups L, Q₁, Q₂, X, Y, R^{a}, R^{b}, R^{c}, R¹, R², R³ and R⁴ have the meanings given in the claims and description, the isomers thereof, processes for preparing these pteridinones and their use as pharmaceutical compositions.

### BACKGROUND TO THE INVENTION

Tumour cells wholly or partly elude regulation and control by the body and are characterised by uncontrolled growth. This is due on the one hand to the loss of control proteins such as for example Rb, p16, p21 and p53 and also to the activation of so-called accelerators of the cell cycle, the cyclin-dependent kinases (CDK's).

Moreover, the protein kinase Aurora B has also been described as having an essential function during entry into mitosis. Aurora B phosphorylates histone H3 on Ser10 and thereby initiates chromosome condensation (Hsu et al. 2000, Cell 102:279-91). A specific cell cycle arrest in the G2/M phase may, however, also be initiated e.g. by inhibition of specific phosphatases such as e.g. Cdc25C (Russell and Nurse 1986, Cell 45:145-53). Yeasts with a defective Cdc25 gene arrest in the G2 phase, whereas overexpression of Cdc25 leads to premature entry into the mitosis phase (Russell and Nurse, 1987, Cell 49:559-67). Moreover, an arrest in the G2/M phase may also be initiated by inhibition of specific motor proteins, the so-called kinesins such as for example Eg5 (Mayer et al. 1999, Science 286:971-4), or by microtubuli stabilising or destabilising agents (e.g. colchicin, taxol, etoposide, vinblastine, vincristine) (Schiff and Horwitz 1980, Proc Natl Acad Sci USA 77:1561-5).

In addition to the cyclin-dependent and Aurora kinases the so-called polo-like kinases, a small family of serine/threonine kinases, also play an important role in the regulation of the eukaryotic cell cycle. Up till now the polo-like kinases PLK-1, PLK-2, PLK-3 and PLK-4 have been described in the literature. PLK-1 in particular has been shown to play a central role in the regulation of the mitosis phase. PLK-1 is responsible for the maturation of the centrosomes, for the activation of phosphatase Cdc25C, as well as for the activation of the Anaphase Promoting Complex (Glover et al. 1998, Genes Dev. 12:3777-87; Qian et al. 2001, Mol Biol Cell. 12:1791-9). The injection of PLK-1 antibodies leads to a G2 arrest in untransformed cells, whereas tumour cells arrest during the mitosis phase (Lane and Nigg 1996, J. Cell Biol. 135:1701-13). Overexpression of PLK-1 has been demonstrated in various types of tumour, such as non-small-cell carcinoma of the lung, plate epithelial carcinoma, breast and colorectal carcinoma (Wolf et al. 1997, Oncogene 14:543-549; Knecht et al. 1999, Cancer Res. 59:2794-2797; Wolf et al. 2000, Pathol. Res. Pract. 196:753-759; Takahashi et al. 2003, Cancer Sci. 94:148-52). Therefore, this category of proteins also presents an interesting point of attack for therapeutic intervention in proliferative diseases (Liu and Erikson 2003, Proc Natl Acad Sci USA 100:5789-5794).

Pteridinone derivatives are known from the prior art as active substances with an antiproliferative activity. WO 01/019825 and WO 03/020722 describe the use of pteridinone derivatives for the treatment of tumoral diseases.

The resistance of many types of tumour demands that new drugs be developed to fight the tumours. The aim of the present invention is therefore to provide new compounds having an antiproliferative activity.

### DETAILED DESCRIPTION OF THE INVENTION

It has now been found that, surprisingly, compounds of general formula (1), wherein the groups L, Q₁, Q₂, X, Y, Z, R^{a}, R^{b}, R^{c}, R¹, R², R³ and R⁴ are defined as hereinafter, act as inhibitors of specific cell cycle kinases. Thus, the compounds according to the invention may be used for example for the treatment of diseases associated with the activity of specific cell cycle kinases and characterised by excessive or anomalous cell proliferation.

The present invention relates to compounds of general formula (1) 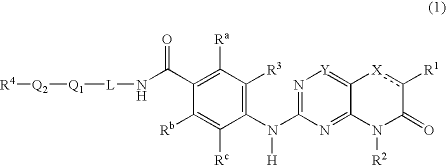
wherein
the dotted line denotes an optional bond, while
X denotes N or C—R^{e}, if X and CR¹ are linked by a double bond, or
X denotes —N—R^{d}, if X and CR¹ are linked by a single bond,
Y denotes N or CH;
R¹ denotes a group selected from among hydrogen, halogen, ═O, —OR⁵, —C(═O)R⁶, —C(═O)NR⁵R⁶, —NR⁵R⁶, —NR⁵C(═O)R⁶, —NR⁵SO₂R⁶, —N═CR⁵R⁶, —SR⁵, —SOR⁵, —SO₂R⁵, —SO₂NR⁵R⁶ and pseudohalogen,
   or an optionally mono- or polysubstituted group selected from among C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵ SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen, or, if X and CR¹ are linked by a single bond, the group ═O as well;
R² denotes hydrogen or an optionally mono- or polysubstituted group selected from among C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
R³ denotes a group selected from among hydrogen, halogen, —OR⁵, —C(═O)R⁵, —C(═O)NR⁵R⁶, —NR⁵R⁶, —NR⁵C(═O)R⁶, —NR⁵SO₂R⁶, —N═CR⁵R⁶, —SR⁵, —SOR⁵, —SO₂R⁵, —SO₂NR⁵R⁶ and pseudohalogen,
   or an optionally mono- or polysubstituted group selected from among C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen:
L denotes a bond or a group selected from among optionally mono- or polysubstituted C₁₋₁₆-alkyl, C₂₋₁₆-alkenyl and C₂₋₁₆-alkynyl, while the substituent(s) may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
Q₁ and Q₂ each independently of one another denote a bond or a group selected from among optionally mono- or polysubstituted C₁₋₁₆-alkyl, C₂₋₁₆-alkenyl, C₂₋₁₆-alkynyl, C₃₋₁₀-cycloalkyl, aryl, heterocyclyl and heteroaryl, while the substituent(s) may be identical or different and are selected from among halogen, —NO₂, R⁵, —OR⁵, —C(═O)R⁵, —C(═O)OR⁵, —C(═O)NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
R⁴ denotes hydrogen or a group selected from among optionally mono- or polysubstituted C₁₋₁₆alkyl, C₂₋₁₆alkenyl, C₂₋₁₆alkynyl, C₃₋₁₀cycloalkyl, aryl, heterocyclyl and heteroaryl, while the substituent(s) may be identical or different and are selected from among —NO₂, R⁵, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵ SO₂NR⁶R⁷, —OSO₂NR⁵R⁶ and pseudohalogen;
R^{a}, R^{b}, R^{c}, each independently of one another denote a group selected from among hydrogen, halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen; or an optionally mono- or polysubstituted group selected from among C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen and pseudohalogen;
R^{d} denotes hydrogen or a group selected from among optionally mono- or polysubstituted C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen; R^{e} denotes a group selected from among hydrogen, halogen, pseudohalogen or a group selected from among optionally mono- or polysubstituted C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
R⁵, R⁶ and R⁷ each independently of one another denote hydrogen or a group selected from among optionally mono- or polysubstituted C₁₋₅alkyl, C₂₋₅alkenyl, C₂₋₅alkynyl, C₃₋₁₀cycloalkyl, aryl, heterocyclyl and heteroaryl, while the substituents may be identical or different and are selected from among C₃₋₁₀cycloalkyl, aryl, heterocyclyl, heteroaryl, halogen, —NO₂, —OR⁸, —C(═O)R⁸, —C(═O)OR⁸, —C(═O)NR⁸R⁹, —NR⁸R⁹, —NR⁸C(═O)R⁹, —NR⁸C(═O)OR⁹, —NR⁸C(═O)NR⁹R¹⁰, —NR⁸C(═O)ONR⁹R¹⁰, —NR⁸SO₂R⁹, —N═CR⁸R⁹, —SR⁸, —SOR⁸, —SO₂R⁸, —SO₂NR⁸R⁹, —NR⁸SO₂NR⁹R¹⁰, —OSO₂NR⁸R⁹ and pseudohalogen; R⁸, R⁹ and R¹⁰ each independently of one another denote hydrogen or a group selected from among optionally substituted C₁₋₈alkyl, C₂₋₈alkenyl, C₂₋₈alkynyl, C₃₋₁₀cycloalkyl, aryl, heterocyclyl and heteroaryl, while the substituent(s) may be identical or different and are selected from among halogen, —NH₂, —OH and pseudohalogen;
   optionally in the form of the tautomers, racemates, enantiomers, diastereomers and mixtures thereof, and optionally the pharmacologically acceptable acid addition salts thereof.

In one aspect the invention relates to compounds of general formula (1), wherein
Y denotes CH.

In another aspect the invention relates to compounds of general formula (1), wherein
R^{c} denotes a group selected from among hydrogen, —F, —Cl, methyl and ethyl.

In another aspect the invention relates to compounds of general formula (1), wherein
R^{a} and R^{b} each independently of one another denote hydrogen or fluorine;
or an optionally mono- or polysubstituted group selected from among C₁₋₂alkyl, C₂alkenyl, C₂alkynyl, C₃₋₆cycloalkyl, aryl, heterocyclyl and heteroaryl, while the substituents may be identical or different and are selected from among hydrogen, halogen, —NO₂, —OR⁴, —C(═O)R⁴, —C(═O)OR⁴, —C(═O)NR⁴R⁵, —NR⁴R⁵, —NR⁴C(═O)R⁵, —NR⁴C(═O)OR⁵, —NR⁴C(═O)NR⁵R⁶, —NR⁴SO₂R⁵, —N═CR⁴R⁵, —SR⁴, —SOR⁵, —SO₂R⁴, —SO₂NR⁴R⁵, —NR⁴, —SO₂NR⁴R⁵, —OSO₂NR⁴R⁵ and pseudohalogen.

In an additional aspect the invention relates to compounds of general formula (1), wherein R^{a} and R^{b} independently of one another represent hydrogen or fluorine and the remaining groups are as hereinbefore defined.

The invention also encompasses compounds of general formula (1) wherein
R² denotes isopropyl or cyclopentyl and the remaining groups are as hereinbefore defined.

In one aspect the invention relates to the use of compounds of general formula (1) as pharmaceutical compositions.

In another aspect the invention relates to the use of compounds of general formula (1) as pharmaceutical compositions with an antiproliferative activity.

In an essential aspect the invention relates to the use of compounds of general formula (1) for preparing a pharmaceutical composition for the treatment and/or prevention of diseases selected from among cancer, bacterial and viral infections, inflammatory and autoimmune diseases, chemotherapy-induced alopecia and mucositis, cardiovascular diseases, nephrological diseases, as well as chronic and acute neurodegenerative diseases.

In another aspect the invention relates to the use of a compound of formula (I) for preparing a pharmaceutical composition for inhibiting the polo-like kinases.

In another aspect the invention relates to the use of a compound of general formula (1) for preparing a pharmaceutical composition for inhibiting the polo-like kinase PLK1.

In one aspect the invention relates to the use of compounds of general formula (1) for preparing a pharmaceutical composition for the treatment and/or prevention of tumour diseases based on the overexpression of the polo-like kinases.

In another aspect the invention relates to a method for the treatment and/or prevention of diseases selected from among cancer, bacterial and viral infections, inflammatory and autoimmune diseases, chemotherapy-induced alopecia and mucositis, cardiovascular diseases, nephrological diseases, as well as chronic and acute neurodegenerative diseases, characterised in that an effective amount of a compound of formula (I) according to one of claims 1 to 6 is administered to a patient.

In an additional aspect the invention relates to pharmaceutical preparations, containing as active substance one or more compounds of general formula (I) according to one of claims 1 to 6 optionally in conjunction with conventional excipients and/or carriers.

Definitions

As used herein, the following definitions apply, unless stated otherwise.

By alkyl substituents are meant in each case saturated, straight-chain or branched aliphatic hydrocarbon groups (alkyl group).

The alkenyl substituents are in each case straight-chain or branched, unsaturated alkyl groups which have at least one double bond.

By alkynyl substituents are meant in each case straight-chain or branched, unsaturated alkyl groups which have at least one triple bond.

Haloalkyl refers to alkyl groups wherein one or more hydrogen atoms are replaced by halogen atoms. Haloalkyl includes both saturated alkyl groups and unsaturated alkenyl and alkynyl groups, such as for example —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CHFCH₂CH₃ and —CHFCH₂CF₃.

Halogen relates to fluorine, chlorine, bromine and/or iodine atoms.

By pseudohalogen are meant the following groups: —OCN, —SCN, —CF₃ and —CN.

By cycloalkyl is meant a mono- or bicyclic ring, while the ring system may be a saturated ring or an unsaturated, non-aromatic ring, which may optionally also contain double bonds, such as for example cyclopropyl, cyclopropenyl, cyclobutyl, cyclobutenyl, cyclopentyl, cyclopentenyl, cyclohexyl, cyclohexenyl, norbornyl, norbornenyl, spiro[5.5]undecane, spiro[5.4]decane and spiro[4.4]nonane.

Aryl relates to monocyclic or bicyclic rings with 6-12 carbon atoms such as for example phenyl and naphthyl.

By heteroaryl are meant mono- or bicyclic rings which contain instead of one or more carbon atoms one or more identical or different heteroatoms, such as e.g. nitrogen, sulphur or oxygen atoms. Examples include furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl and triazinyl. Examples of bicyclic heteroaryl groups are indolyl, isoindolyl, benzofuranyl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl and benzotriazinyl, indolizinyl, oxazolopyridinyl, imidazopyridinyl, naphthyridinyl, indolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, cumarinyl, isocumarinyl, chromonyl, chromanonyl, pyridinyl-N-oxide tetrahydroquinolinyl, dihydroquinolinyl, dihydroquinolinonyl, dihydroisoquinolinonyl, dihydrocumarinyl, dihydroisocumarinyl, isoindolinonyl, benzodioxanyl, benzoxazolinonyl, pyrrolyl-N-oxide, pyrimidinyl-N-oxide, pyridazinyl-N-oxide, pyrazinyl-N-oxide, quinolinyl-N-oxide, indolyl-N-oxide, indolinyl-N-oxide, isoquinolyl-N-oxide, quinazolinyl-N-oxide, quinoxalinyl-N-oxide, phthalazinyl-N-oxide, imidazolyl-N-oxide, isoxazolyl-N-oxide, oxazolyl-N-oxide, thiazolyl-N-oxide, indolizinyl-N-oxide, indazolyl-N-oxide, benzothiazolyl-N-oxide, benzimidazolyl-N-oxide, pyrrolyl-N-oxide, oxadiazolyl-N-oxide, thiadiazolyl-N-oxide, triazolyl-N-oxide, tetrazolyl-N-oxide, benzothiopyranyl-S-oxide and benzothiopyranyl-S,S-dioxide.

Heterocyclyl relates to saturated or unsaturated, non-aromatic mono-, bicyclic or bridged bicyclic rings comprising 5-12 carbon atoms, which carry heteroatoms, such as nitrogen, oxygen or sulphur, instead of one or more carbon atoms. Examples of such heterocyclyl groups are tetrahydrofuranyl, pyrrolidinyl, pyrrolinyl, imidazolidinyl, imidazolinyl, pyrazolidinyl, pyrazolinyl, piperidyl, piperazinyl, indolinyl, isoindoliny, morpholinyl, thiomorpholinyl, homomorpholinyl, homopiperidyl, homopiperazinyl, thiomorpholinyl-S-oxide, thiomorpholinyl-S,S-dioxide, tetrahydropyranyl, piperidinyl, tetrahydrothienyl, homopiperidinyl, homothiomorpholinyl-S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl-S-oxide, tetrahydrothienyl-S,S-dioxide, homothiomorpholinyl-S-oxide, 2-oxa-5-azabicyclo[2.2.1]heptane, 8-oxa-3-azabicyclo[3.2.1]octane, 3,8-diaza-bicyclo[3.2.1]octane, 2,5-diaza-bicyclo[2.2.1]heptane, 3,8-diaza-bicyclo[3.2.1]octane, 3,9-diaza-bicyclo[4.2.1]nonane and 2,6-diaza-bicyclo[3.2.2]nonane, 2,7-diaza-spiro[3.5]nonane, 2,7-diaza-spiro[4.4]nonane, 2,8-diaza-spiro[4.5]decane and 3,9-diaza-spiro[5.5]undecane.

Preparation of the Compounds According to the Invention:

The compounds according to the invention may be prepared according to methods of synthesis A to C described hereinafter, wherein the substituents of general formulae (I to XVI) have the meanings given hereinbefore. These methods are to be understood as being an illustration of the invention without restricting it to their content.

Analysis

Preparative Chromatography:

For medium pressure chromatography (MPLC) silica gel made by Millipore (name: Granula Silica Si-60A 35-70 μm) or C-18 RP-silica gel made by Macherey Nagel (name: Polygoprep 100-50 C18) is used.

For preparative high pressure chromatography columns made by Waters (name: XTerra Prep. MS C18, 5 μM, 30*100 mm or Symmetrie C18, 5 μm, 19*100) are used.

Nuclear Magnetic Resonance (NMR) Spectroscopy:

The measurement is carried out in deuterised dimethylsulphoxide-d6. If other solvents are used they are explicitly mentioned in the Examples or in the methods. The measurements are given on a delta scale in ppm. Tetramethylsilane is taken as the standard. The measurements are carried out on an Avance 400 (400 MHz NMR spectrometer) made by Messrs Bruker Biospin GmbH.

The NMR spectra are given purely in a descriptive capacity. Basically, only the visible molecular signals are listed. If for example molecular signals are partly or completely masked by foreign signals such as for example water signals, DMSO signals or CDCl₃ signals they are not mentioned.

Mass Spectroscopy/UV Spectrometer:

These data are generated using an HPLC-MS apparatus (high performance liquid chromatography with mass detector) made by Agilent.

The apparatus is constructed so that a diode array detector (G1315B made by Agilent) and a mass detector (1100 LS-MSD SL; G1946D; Agilent) are connected in series downstream of the chromatography apparatus (column: Zorbax SB-C8, 3.5 μm, 2.1*50, Messrs. Agilent). The apparatus is operated with a flow of 0.6 ml/min. For a separation process a gradient is run through within 3.5 min (start of gradient: 95% water and 5% acetonitrile; end of gradient: 5% water and 95% acetonitrile; in each case 0.1% formic acid is added to the two solvents).

Method A

Step 1A

The intermediate compound III is prepared by substitution of a leaving group LG, for example halogen, SCN, methoxy, methanesulphonyl, preferably methanesulphinyl or chlorine, on a heteroaromatic system I by a nucleophile II.

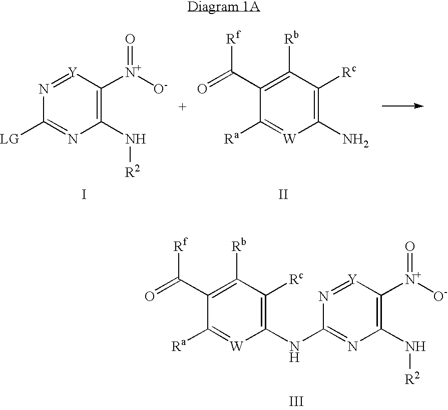

The group R^{f} either corresponds to NH-L-Q₁-Q₂-R⁴ or denotes benzyloxy, methoxy or hydroxy.

1 equivalent of the compound I and 1 to 2 equivalents of the compound II are stirred in a solvent, for example 1,4-dioxane, tetrahydrofuran, N,N-dimethylformamide or N,N-dimethylacetamide.

The reaction mixture is stirred for another 1 to 5 days at a temperature of 15-25° C. Then the solvent is distilled off and the residue is purified by chromatography.

Step 2A

The intermediate compound IV is prepared by reduction of the nitro group on a heteroaromatic system III.

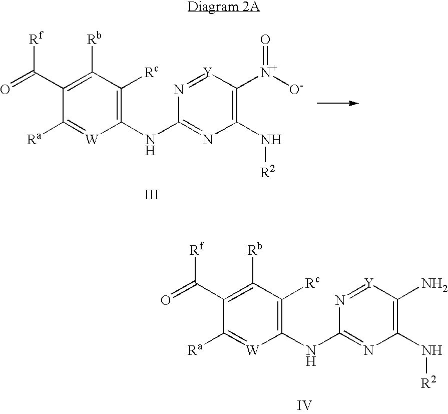

The compound III is dissolved in a solvent, for example methanol, ethanol, N,N-dimethylformamide, ethyl acetate, tetrahydrofuran or acetone. A catalyst is added, for example palladium on charcoal, palladium hydroxide or Raney nickel. This suspension is transferred into an autoclave. This is subjected to a hydrogen pressure of 2 to 10 bar. The mixture is stirred for 1 to 5 days at 20 to 60° C. Then the catalyst is filtered off and the solvent is eliminated in vacuo.

Alternatively the above solution may also be combined with tin (II)chloride and stirred for 0.5-10 h at 30 to 100° C. After aqueous working up the organic phase is evaporated down in vacuo.

Step 3A

The intermediate compound VI is prepared by condensation of a glyoxylate derivative V with a compound IV.

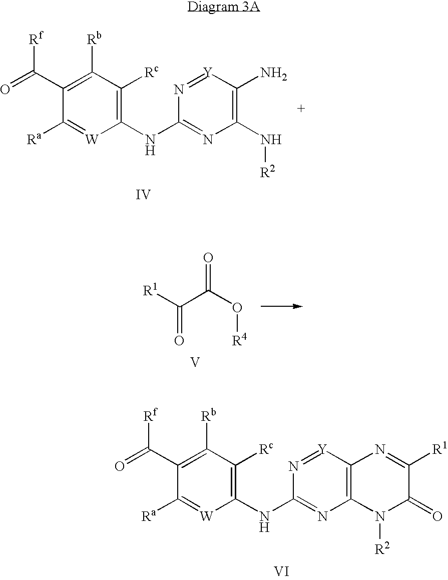

1 equivalent of the compound IV and 1 to 2 equivalents of the compound V are stirred in a solvent, for example 1,4-dioxane, tetrahydrofuran, N,N-dimethylformamide, ethanol, methanol or N,N-dimethylacetamide. At a temperature of 15 to 40° C. 3 to 7 equivalents of a Brönsted acid or Lewis acid, for example sulphuric acid, acetic acid, formic acid, hydrochloric acid, aluminium trichloride, titanium tetrachloride or ytterbium(III)triflate hydrate are added. The reaction mixture is stirred for another 6 to 36 h at a temperature of 50 to 150° C. Then the solvent is distilled off and the residue is purified by chromatography.

Step 4A

Compounds VI whose group R^{f} denotes hydroxy may be used directly for preparing the end compounds VIII, by reacting a compound VI with a compound VII.

Compounds VI having a group R^{f} which does not denote hydroxy are converted beforehand by hydrolysis or similar methods known to the skilled man into the compounds wherein the group R^{f} denotes hydroxy.

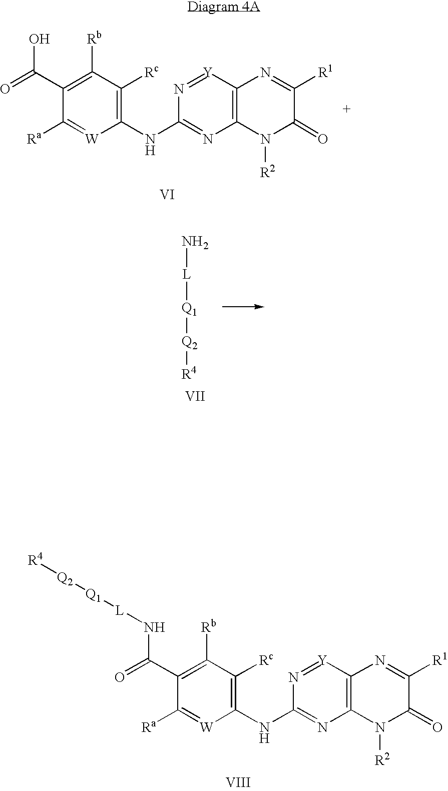

1 equivalent of the compound VI, 1 to 1.5 equivalents of the compound VII and 1 to 3 equivalents of a base, for example triethylamine or ethyldiisopropylamine, are stirred in a solvent, for example 1,4-dioxane, N,N-dimethylformamide, N,N-dimethylacetamide or N-methyl-2-pyrrolidinone. At a temperature of 15 to 25° C. 1 to 1.5 equivalents of a coupling reagent, for example N,N-dicyclohexylcarbodiimide, N,N-diisopropylcarbodiimide, O-(benzotriazol-1-yl)-N,N,N′,N′-tetramethyluronium-tetrafluoroborate or 1-(3-N,N-dimethylaminopropyl)-3-ethylcarbodiimide are added. The reaction mixture is stirred for 4 to 24 h at a temperature of 15 to 25° C. Then the solvent is distilled off and the residue is purified by chromatography.

Method B

Step 1B

The intermediate compound III is prepared by substitution of a leaving group LG, for example halogen, SCN, methoxy, methanesulphonyl, preferably methanesulphinyl or chlorine, on a heteroaromatic system I by a nucleophile II.

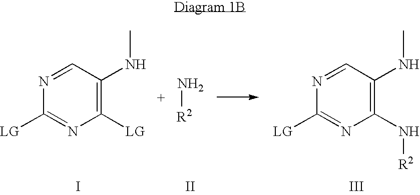

1 equivalent of the compound I and 1 to 1.5 equivalents of the compound II are stirred in a solvent, for example 1,4-dioxane, tetrahydrofuran, ethyl acetate, N,N-dimethylformamide, acetonitrile or N,N-dimethylacetamide.

At a temperature of 15 to 25° C. 2 to 2.5 equivalents of a base such as potassium carbonate, sodium carbonate, caesium carbonate, N-ethyl-N,N-diisopropylamine or triethylamine are added. The reaction mixture is stirred for another 12 to 72 h at a temperature of 15 to 25° C. The insoluble ingredients are filtered off and washed with one of the above-mentioned solvents. Then the solvent is distilled off and the residue is purified by chromatography.

Step 2B

The intermediate compound VI is prepared by condensation of an oxalic acid derivative with an intermediate compound IV.

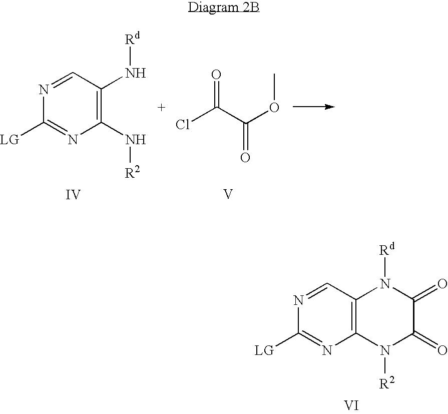

1 equivalent of the compound IV and 2 to 2.5 equivalents of a base such as potassium carbonate, sodium carbonate, caesium carbonate, N-ethyl-N,N-diisopropylamine or triethylamine are stirred in a solvent, for example 1,4-dioxane, toluene, tetrahydrofuran, ethyl acetate, N,N-dimethylformamide, acetonitrile or N,N-dimethylacetamide. At a temperature of −78 to −30° C. 1 to 1.5 equivalents of a compound V are added. The reaction mixture is stirred for a further 3 to 6 h at a temperature of −70 to −30° C. Then the mixture is left to warm up to 20° C. within 5 to 12 h and stirred for 6 to 12 h at 130° C. After filtration of the reaction solution through silica gel the solvent is eliminated in vacuo and the residue is recrystallised from water.

Step 3B

The compound VIII is prepared by substitution of a leaving group LG, for example halogen, SCN, methoxy, methanesulphonyl, preferably methanesulphinyl or chlorine on a heteroaromatic system VI by a nucleophile VII.

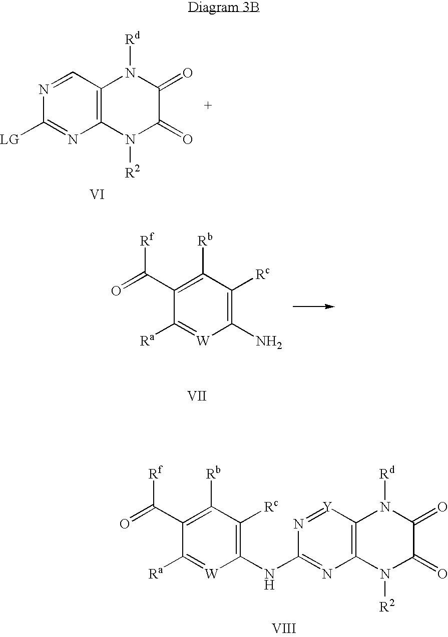

1 equivalent of the compound VI and 1 to 3 equivalents of the compound VII are stirred in a solvent, for example 1,4-dioxane, N,N-dimethylformamide, N,N-dimethylacetamide or N-methyl-2-pyrrolidinone. At a temperature of 15 to 40° C. 1 to 10 equivalents of an inorganic acid, for example sulphuric acid or hydrochloric acid are added. The reaction mixture is stirred for a further 12 to 72 h at a temperature of 60 to 120° C. Then the solvent is distilled off and the residue is purified by chromatography.

Step 4B

Compounds VIII whose group R^{f} denotes hydroxy may be used directly to prepare the end compounds X, by reacting a compound VIII with a compound IVX.

Compounds VIII having a group R^{f} which does not denote hydroxy are converted beforehand into the compounds wherein the group R^{f} denotes hydroxy by hydrolysis or similar methods known to the skilled man.

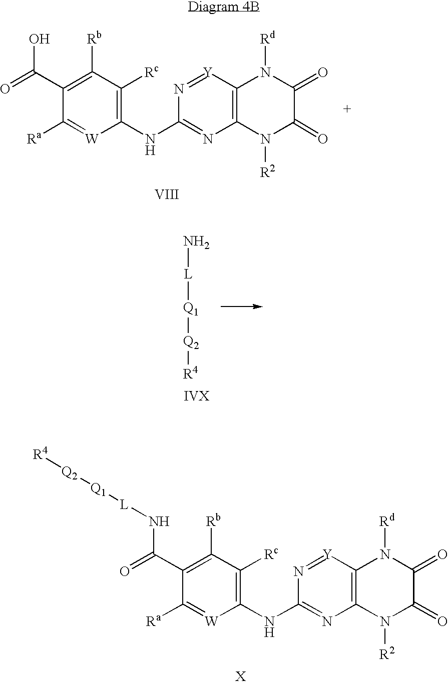

1 equivalent of the compound VIII, 1 to 1.5 equivalents of the compound IVX and 1 to 3 equivalents of a base such as triethylamine or ethyldiisopropyl-amine are stirred in a solvent, for example 1,4-dioxane, N,N-dimethylformamide, N,N-dimethylacetamide or N-methyl-2-pyrrolidinone. At a temperature of 15 to 25° C. 1 to 1.5 equivalents of a coupling reagent, for example N,N-dicyclohexylcarbodiimide, N,N-diisopropylcarbodiimide, O-(benzotriazol-1-yl)-N,N,N′,N′-tetramethyluronium-tetrafluoroborate or 1-(3-N,N-dimethylaminopropyl)-3-ethylcarbodiimide are added. The reaction mixture is stirred for a further 4 to 24 h at a temperature of 15 to 25° C. Then the solvent is distilled off and the residue is purified by chromatography.

Method C

Step 1C

The compound I is prepared as described in WO0119825.

The compound III is prepared by substitution of a leaving group LG, for example halogen, SCN, methoxy, methanesulphonyl, preferably methanesulphinyl or chlorine, on a heteroaromatic system I by a nucleophile II.

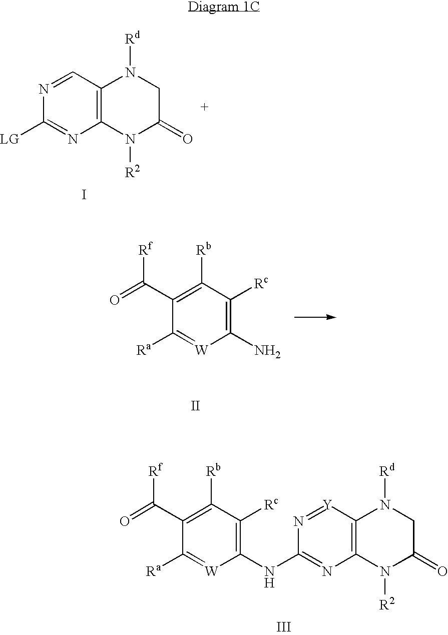

The preparation is carried out analogously to WO0119825.

Step 2C

Compounds III whose group R^{f} denotes hydroxy may be used directly to prepare the end compounds V, by reacting a compound III with a compound IV.

Compounds III having a group R^{f} which does not denote hydroxy are converted beforehand into the compounds wherein the group R^{f} denotes hydroxy by hydrolysis or similar methods known to the skilled man.

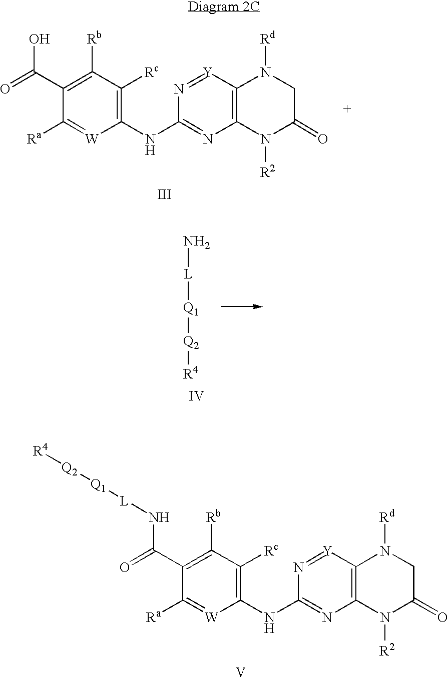

1 equivalent of the compound III, 1 to 1.5 equivalents of the compound IV and 1 to 3 equivalents of a base such as triethylamine or ethyldiisopropylamine are stirred in a solvent, for example 1,4-dioxane, N,N-dimethylformamide, N,N-dimethylacetamide or N-methyl-2-pyrrolidinone. At a temperature of 15 to 25° C. 1 to 1.5 equivalents of a coupling reagent, for example N,N-dicyclohexylcarbodiimide, N,N-diisopropylcarbodiimide, O-(benzotriazol-1-yl)-N,N,N′,N′-tetramethyluronium-tetrafluoroborate or 1-(3-N,N-dimethylaminopropyl)-3-ethylcarbodiimide are added. The reaction mixture is stirred for a further 4 to 24 h at a temperature of 15 to 25° C. Then the solvent is distilled off and the residue is purified by chromatography.

Method D

Step 1D

The compound I is prepared as described in WO0170741.

The compound III is prepared by substitution of a leaving group LG, for example halogen, SCN, methoxy, methanesulphonyl, preferably methanesulphinyl or chlorine, on a heteroaromatic system I by a nucleophile II.

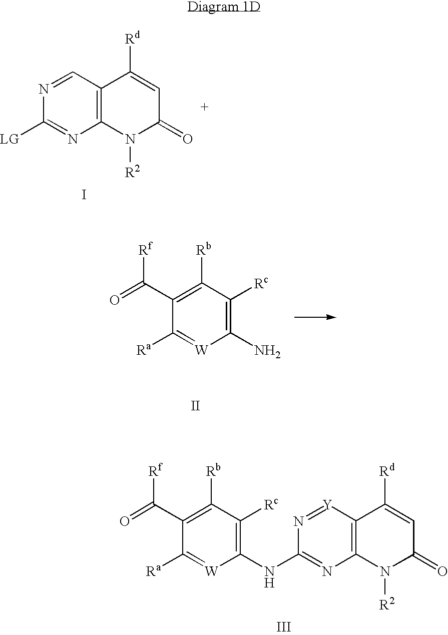

The preparation is carried out analogously to WO0170741.

Step 2D

Compounds III whose group R^{f} denotes hydroxy may be used directly to prepare the end compounds V, by reacting a compound III with a compound IV.

Compounds III having a group R^{f} which does not denote hydroxy are converted beforehand into the compounds wherein the group R^{f} denotes hydroxy by hydrolysis or similar methods known to the skilled man.

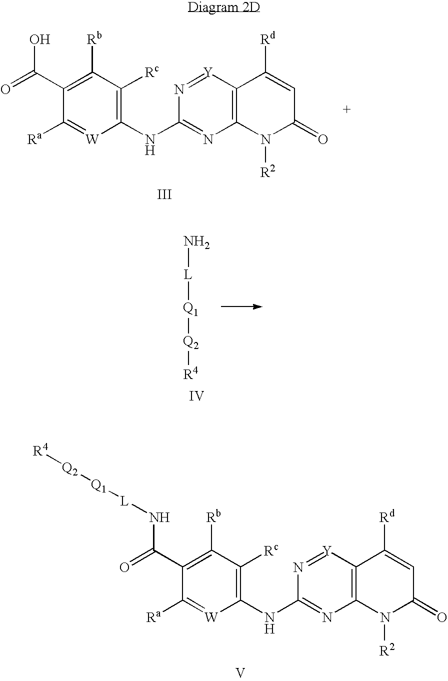

1 equivalent of the compound III, 1 to 1.5 equivalents of the compound IV and 1 to 3 equivalents of a base such as triethylamine or ethyldiisopropylamine are stirred in a solvent, for example 1,4-dioxane, N,N-dimethylformamide, N,N-dimethylacetamide or N-methyl-2-pyrrolidinone. At a temperature of 15 to 25° C. 1 to 1.5 equivalents of a coupling reagent, for example N,N-dicyclohexylcarbodiimide, N,N-diisopropylcarbodiimide, O-(benzotriazol-1-yl)-N,N,N′,N′-tetramethyluronium-tetrafluoroborate or 1-(3-N,N-dimethylaminopropyl)-3-ethylcarbodiimide are added. The reaction mixture is stirred for a further 4 to 24 h at a temperature of 15 to 25° C. Then the solvent is distilled off and the residue is purified by chromatography.

Method 1

### 4-(8-cyclopentyl-5-methyl-6,7-dioxo-5,6,7,8-tetrahydro-pteridin-2-ylamino)-benzoic acid

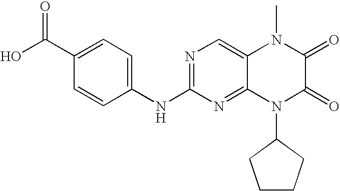

### a) 2-chloro-N⁴-cyclopentyl-N⁵-methyl-pyrimidine-4,5-diamine

4.0 g (22.5 mmol) 2,4-dichloro-5-methylamino-pyrimidine and 6.4 g (46.1 mmol) potassium carbonate are placed in 100 ml acetonitrile. Then 2.5 ml (24.7 mmol) cyclopentylamine are added and the mixture is stirred for 3 days at 25° C. The insoluble ingredients are filtered off. The filtrate is evaporated down in vacuo and the crude product is purified by column chromatography. Silica gel is used as carrier and the eluant used is a mixture of cyclohexane and ethyl acetate (2:1).

Yield: 1.6 g (7.1 mmol, 31%)

MS (ESI): 227 (M+H)⁺

### b) 2-chloro-8-cyclopentyl-5-methyl-5,8-diydropteridine-6,7-dione

2.4 g (10.6 mmol) 2-chloro-N⁴-cyclopentyl-N⁵-methyl-pyrimidine-4,5-diamine in 80 ml of toluene and 0.08 ml N,N-dimethylacetamide are combined with 5.6 ml (32.3 mmol) N,N-diisopropylethylamine and then with 1.5 ml (16.1 mmol) methyloxalyl chloride under an argon atmosphere and at −65° C. The mixture is stirred for 3 h at this temperature and then left to warm up to 25° C. within 4 h. Then it is refluxed for 3 h. The reaction mixture is filtered through silica gel, washed with toluene and then evaporated down in vacuo. The residue is stirred with water, suction filtered, was washed with water and then with ether and dried in vacuo.

Yield: 2.33 g (8.3 mmol, 78%)

MS (ESI): 303 (M+Na)⁺

### c) 4-(8-cyclopentyl-5-methyl-6,7-dioxo-5.6.7,8-tetrahydro-pteridin-2-ylamino)-benzoic acid

33 mg (0.12 mmol) 2-chloro-8-cyclopentyl-5-methyl-5,8-diydropteridin-6,7-dione are dissolved in 1 ml dioxane and combined with 21 mg 4-aminobenzoic acid and 0.47 ml (0.94 mmol) 2 N hydrochloric acid. The mixture is refluxed for 36 h. Then the reaction mixture is made basic with sodium hydrogen carbonate and evaporated down. The crude product is purified by column chromatography. Silica gel is used as carrier and ethanol is used as eluant.

Yield: 14 mg (0.04 mmol, 31%)

MS (ESI): 404 (M+Na)⁺

Method 2

### 4-(4-amino-cyclohexyl)-morpholine

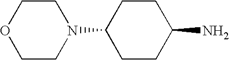

### a) dibenzyl-(4-morpholino-4-yl-cyclohexyl)-amine

3.9 g (30 mmol) 4-dibenzylamino-cyclohexanone are dissolved in 100 ml dichloromethane and stirred with 3.9 g (45 mmol) morpholine and 9.5 g (45 mmol) sodium triacetoxyborohydride for 12 h at ambient temperature. Then water and potassium carbonate are added, the organic phase is separated off, dried and the solvent is eliminated in vacuo. The crude product is purified by column chromatography. Silica gel is used as carrier and the eluant used is ethyl acetate, to which 10% of a mixture of 90% methanol and 10% saturated aqueous ammonia solution have been added. The desired fractions are collected and evaporated down in vacuo.

Yield: 6.6 g (18 mmol, 60%) cis-isomer

2 g (5.4 mmol, 18%) trans-isomer

### b) trans-4-morpholino-4-yl-cyclohexylamine

7.2 g (16.4 mmol) trans-dibenzyl-4-morpholino-cyclohexylamine are dissolved in 100 ml of methanol and hydrogenated on 1.4 g palladium on charcoal (10% Pd) at 30 to 50° C. The solvent is eliminated in vacuo and the residue is crystallised from ethanol and conc. HCl.

Yield: 3.9 g (15.2 mmol, 93%)

melting point: 312° C.

The following compounds are prepared analogously to this method. The amines used are commercially obtainable or their preparation is known from the literature (J Chem Soc 1948, 155, 157).

| | MS |
|---|---|
| | (ESI) |
| | (M + H)⁺: |
| 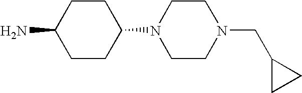 | 238 |
| 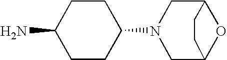 | 211 |

Method 3

### 4-(8-cyclopentyl-6-methyl-7-oxo-7,8-dihydro-pteridin-2-ylamino)-3-methoxy-benzoic acid

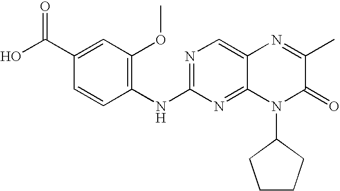

### a) benzyl 4-(4-cyclopentylamino-5-nitro-pyrimidine-2-ylamino)-3-methoxy-benzoate

2.4 g (9.8 mmol) (2-chloro-5-nitro-pyrimidine-4-yl)-cyclopentyl-amine and 4.36 g (14.8 mmol) benzyl 4-amino-3-methoxybenzoate are suspended in 12 ml isopropanol and refluxed for 1 h. The reaction mixture is cooled to 20° C. and the resulting precipitate is suction filtered. This crude product is purified by column chromatography. Silica gel is used as carrier and the eluant used is a mixture of cyclohexane and ethyl acetate (8:2).

Yield: 2.4 g (5.1 mmol, 52%)

MS (ESI): 464 (M+H)⁺

### b) benzyl 4-(4-cyclopentylamino-5-amino-pyrimidine-2-ylamino)-3-methoxy-benzoate

2.4 g (5.1 mmol) benzyl 4-(4-cyclopentylamino-5-nitro-pyrimidine-2-ylamino)-3-methoxy-benzoate are heated to 70° C. in 150 ml of ethyl acetate and combined with 3.5 g (15.5 mmol) tin(II)chloride dihydrate. The mixture is stirred for 30 min at this temperature. Then the reaction mixture is left to cool to 20° C. and 3 ml concentrated ammonia are added. The insoluble ingredients are filtered off and the organic phase is separated off. The organic phase is dried and evaporated down in vacuo.

Yield: 1.9 g (4.4 mmol, 86%)

MS (ESI): 434 (M+H)⁺

### c) benzyl 4-(8-cyclopentyl-6-methyl-7-oxo-7.8-dihydro-pteridin-2-ylamino)-3-methoxy-benzoate

300 mg (0.69 mmol) benzyl 4-(4-cyclopentylamino-5-amino-pyrimidine-2-ylamino)-3-methoxy-benzoate, 77 μl (0.69 mmol) ethyl 2-oxo-propionate and 71 mg (0.69 mmol) ytterbium triflate monohydrate are dissolved in 6 ml dioxane. After 6 h at 120° C. the solvent is eliminated in vacuo. The crude product is purified by column chromatography. The carrier material used is C 18-RP-silica gel and a gradient is run through which consists of 95% water and 5% acetonitrile at the starting point and 2% water and 98% acetonitrile at the finishing point. 0.1% formic acid is added to both solvents.

Yield: 80 mg (0.17 mmol, 24%)

MS (ESI): 486 (M+H)⁺

### d) 4-(8-cyclopentyl-6-methyl-7-oxo-7.8-dihydro-pteridin-2-ylamino)-3-methoxy-benzoic acid

80 mg (0.165 mmol) benzyl 4-(8-cyclopentyl-6-methyl-7-oxo-7.8-dihydro-pteridin-2-ylamino)-3-methoxy-benzoate are dissolved in 440 μl 5 M dioxanic hydrochloric acid and in 310 μl 6 M aqueous hydrochloric acid and refluxed for 16 h. The resulting precipitate is filtered off and dried.

Yield: 60 mg (0.152 mmol, 92%)

MS (ESI): 396 (M+H)⁺

Method 4

### (S)-1-(tetrahydro-pyran-4-yl)-pyrrolidin-3-ylamine

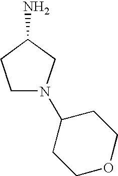

### a) tert-butyl [(S)-1-(tetrahydro-pyran-4-yl)-pyrrolidin-3-yl]-carbamate

4 g (21.47 mmol) (S)-3-Boc-amino-pyrrolidine and 2.37 g (23.62 mmol) tetrahydro-pyran-4-one are dissolved in 50 ml dichloromethane and combined with 0.43 ml (7.51 mmol) glacial acetic acid. The mixture is stirred for 1 h at 25° C. and then 0.8 ml (13.96 mmol) glacial acetic acid and batchwise 9.1 g (42.94 mmol) sodium trisacetoxyborohydride are added. After 2 h at 25° C. the mixture is combined with 50 ml saturated sodium hydrogen carbonate solution. This mixture is stirred for 4 h at 25° C. Then the phases are separated. The aqueous phase is again extracted with 50 ml dichloromethane. The combined organic phases are dried and the solvent is eliminated in vacuo.

Yield: 5.72 g (21.19 mmol, 98%)

### b) (S)-1-(tetrahydro-pyran-4-yl)-pyrrolidin-3-ylamine

11.66 g (43.12 mmol) tert-butyl [(S)-1-(tetrahydro-pyran-4-yl)-pyrrolidin-3-yl]-carbamate are dissolved in 80 ml trifluoroacetic acid while cooling with water. The mixture is stirred for 1 h at 25° C. and the solvent is then eliminated in vacuo. The residue is dissolved in a mixture of ether and isopropanol. This solution is combined with isopropanolic hydrochloric acid. A precipitate settles out. This is suction filtered and dried.

Yield: 9.47 g (38.97 mmol, 90%)

MS (ESI): 171 (M+H)⁺

Method 5

### 4-(8-cyclopentyl-5-methyl-7-oxo-7.8-dihydro-pyrido[2,3-d]pyrimidin-2-ylamino)-benzoic acid

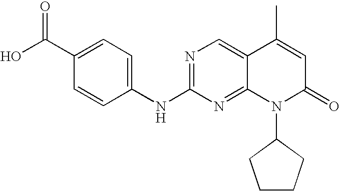

### 8-cyclopentyl-2-methanesulphonyl-5-methyl-8H-pyrido[2,3-d]pyrimidin-7-one

346 mg (1.26 mmol) 8-cyclopentyl-5-methyl-2-methylsulphanyl-8H-pyrido[2,3-d]pyrimidin-7-one (WO 01/70741) are dissolved in 5 ml DCM, combined with 844 mg (3.764 mmol) m-chloroperbenzoic acid (77%) and stirred for 1 h at RT. The reaction mixture is diluted with 120 ml DCM, washed several times with saturated NaHCO₃ solution, dried through MgSO₄, filtered and the solvent is eliminated in vacuo.

Yield: 304 mg (0.99 mmol, 79%)

M (ESI)=308 (M+H)⁺

### Methyl 4-(8-cyclopentyl-5-methyl-7-oxo-7.8-dihydro-pyrido[2,3-d]pyrimidin-2-ylamino)-benzoate

210 mg (0.68 mmol) 8-cyclopentyl-2-methanesulphonyl-5-methyl-8H-pyrido[2,3-d]pyrimidin-7-one and 188 mg (1.23 mmol) methyl 4-aminobenzoate are suspended in 1 ml 2-butanol (anhydrous), combined with 51 μl (0.21 mmol) 4N HCl in 1,4-dioxane and heated to 150° C. for 0.5 h with stirring using a microwave. The solvent is eliminated in vacuo and the residue is purified by column chromatography. The carrier material used is C18-RP-silica gel and a gradient is run through within 20 min which consists of 95% water and 5% acetonitrile at the starting point and 5% water and 95% acetonitrile at the finishing point. 0.2% formic acid are added to both the water and the acetonitrile.

Yield: 91 mg (0.24 mmol, 35%)

M (ESI)=379 (M+H)⁺

### 4-(8-cyclopentyl-5-methyl-7-oxo-7.8-dihydro-pyrido[2,3-d]pyrimidin-2-ylamino)-benzoic acid

271 mg (0.716 mmol) methyl 4-(8-cyclopentyl-5-methyl-7-oxo-7.8-dihydro-pyrido[2,3-d]pyrimidin-2-ylamino)-benzoate are suspended with 175 mg (7.16 mmol) LiOH in a mixture of 5 ml THF, 2 ml MeOH and 2 ml of water and stirred for 3 h at 50° C. Then the reaction mixture is evaporated down to 5 ml, diluted with 10 ml of water, washed 2× with DCM, adjusted to pH 2 with conc. HCl and extracted several times with EE. The combined extracts are dried through MgSO₄, filtered and the solvent is eliminated in vacuo.

Yield: 217 mg (0.60 mmol, 83%)

M (ESI)=365 (M+H)⁺

The following compounds are prepared analogously to this method:

### 4-(8-cyclopropylmethyl-5-methyl-7-oxo-7.8-dihydro-pyrido[2,3-d]pyrimidin-2-ylamino)-benzoic acid

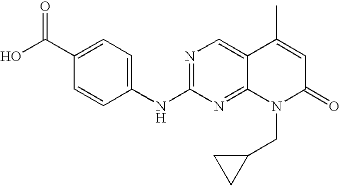

MS (ESI): 351 (M+H)⁺

### 4-(6-acetyl-8-cyclopropylmethyl-5-methyl-7-oxo-7.8-dihydro-pyrido[2,3-d]pyrimidin-2-ylamino)-benzoic acid

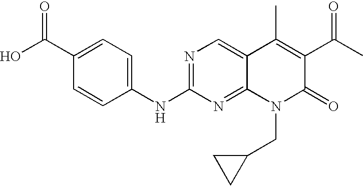

MS (ESI): 393 (M+H)⁺

### 4-(6-acetyl-8-cyclopentyl-5-methyl-7-oxo-7.8-dihydro-pyrido[2,3-d]pyrimidin-2-ylamino)-benzoic acid

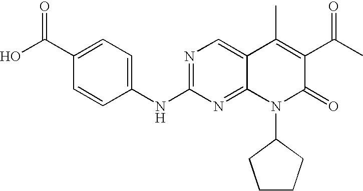

MS (ESI): 407 (M+H)⁺

Method 6

### 4-(4-amino-cyclohexyl)-morpholine

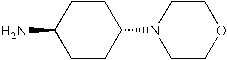

### Dibenzyl-(4-morpholino-4-yl-cyclohexyl)-amine

3.9 g (30 mmol) 4-dibenzylamino-cyclohexanone are dissolved in 100 ml dichloromethane and stirred with 3.9 g (45 mmol) morpholine and 9.5 g (45 mmol) sodium triacetoxy-borohydride for 12 h at RT. Then water and potassium carbonate are added, the organic phase is separated off and dried and the solvent is eliminated in vacuo. The crude product is purified by column chromatography. Silica gel is used as carrier and the eluant used is ethyl acetate, to which 10% of a mixture of 90% methanol and 10% saturated aqueous ammonia solution have been added. The desired fractions were evaporated down in vacuo.

Yield: 6.6 g (18 mmol, 60%) cis-isomer

2 g (5.4 mmol, 18%) trans-isomer.

### trans-4-morpholino-4-yl-cyclohexylamine

7.2 g (16.4 mmol) trans-dibenzyl-4-morpholino-cyclohexylamine were dissolved in 100 ml MeOH and hydrogenated on 1.4 g Pd/C (10%) at 30-50° C. The solvent was eliminated in vacuo and the residue was crystallised from ethanol and conc. HCl.

Yield: 3.9 g (15.2 mmol, 93%)

melting point: 312° C.

### EXAMPLE 1

### 4-(8-cyclopentyl-5-methyl-6,7-dioxo-5,6,7,8-tetrahydro-pteridin-2-ylamino)-N-(4-morpholin-4-yl-cyclohexyl)-benzamide

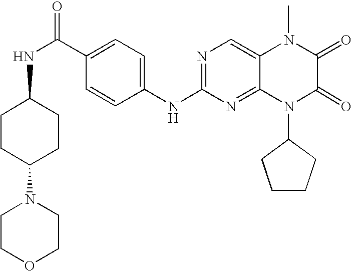

45 mg (0.119 mmol) 4-(8-cyclopentyl-5-methyl-6,7-dioxo-5,6,7,8-tetrahydro-pteridin-2-ylamino)-benzoic acid (method 1), 166 μl (0.954 mmol) N-ethyldiisopropylamine, 46 mg (0.143 mmol) O-(benzotriazol-1-yl)-N,N,N′,N′-tetramethyluronium-tetrafluoroborate and 33 mg (0.179 mmol) trans-4-morpholin-4-yl-cyclohexylamine (method 2) are dissolved in 4 ml N,N-dimethylformamide. After 15 h at ambient temperature the solvent is eliminated in vacuo. The crude product is purified by column chromatography. The carrier material used is C18-RP-silica gel and a gradient is run through which consists of 95% water and 5% acetonitrile at the starting point and 2% water and 98% acetonitrile at the finishing point. 0.1% formic acid is added to both solvents. The compound is obtained as the formate.

Yield: 34 mg (0.061 mmol; 51%)

UV max: 314 nm

MS (ESI): 548 (M+H)⁺

¹H-NMR: 1.23-1.41 (m, 4H), 1.58-1.68 (m, 2H), 1.80-1.93 (m, 6H), 1.94-2.03 (m, 2H), 2.13-2.23 (m, 2H), 5.71 (m, 1H), 7.74-7.82 (m, 4H), 7.97-8.01 (m, 1H), 8.18 (s, 1H), 8.54 (s, 1H), 9.86 (s, 1H)

### EXAMPLES 2-10

The following compounds are prepared by an analogous method to the one described in Example 1. The amine used to prepare the amide is commercially obtainable or may be prepared by the processes described in method 2 or method 4.

In the following Tables x₁ and x₂ denote the point of attachment of the particular fragment of the structure to the generic structural unit.

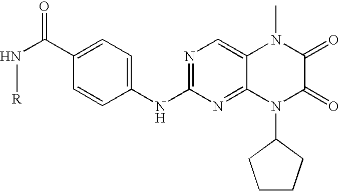

| | | | UV | MS | |
|---|---|---|---|---|---|
| | | | max | (ESI) | |
| # | R—X₁ | salt | [nm] | (M + H)⁺ | NMR |
| 2 | 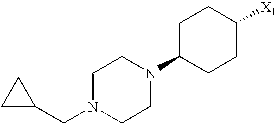 | HCOOH | 310 | 601 | 0.04-0.11 (m, 2H), 0.42-0.5 (m, 2H), 0.77-0.86 (m, 1H), 1.26-1.40 (m, 4H), 1.57-1.68 (m, 2H), 1.80-1.94 (m, 6H), 1.94-2.02 (m, 2H), 2.13-2.23 (m, 4H), 5.71 (m, 1H), 7.75-7.85 (m, 4H), 7.98-8.02 (d, 1H), 8.54 (s, 1H), 9.86 (s, 1H) |
| 3 | 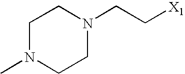 | HCOOH | 318 | 507 | 1.58-1.67 (m, 2H), 1.81-1.89 (m, 2H), 1.94-2.03 (m, 2H), 2.13-2.23 (m, 5H), 2.29-2.39 (m, 4H), 2.40-2.47 (m, 4H), 5.71 (m, 1H), 7.79 (m, 4H), 8.21 (m, 2H), 8.54 (s, 1H), 9.91 (s, 1H) |
| 4 | 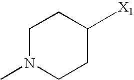 | HCOOH | 314 | 478 | 1.57-1.66 (m, 4H), 1.73-1.79 (m, 2H), 1.81-1.90 (m, 2H), 1.90-2.01 (m, 4H), 2.13-2.20 (m, 5H), 2.75-2.79 (m, 2H), 3.49 (s, 3H), 3.68-3.76 (m, 1H), 5.71 (m, 1H), 7.76-7.82 (m, 4H), 8.02-8.04 (d, 1H), 8.53 (s, 1H), 9.87 (s, 1H) |
| 5 | 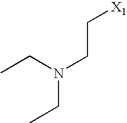 | HCOOH | 310 | 480 | 0.99 (t, 6H), 1.57-1.68 (m, 2H), 1.80-1.91 (m, 2H), 1.92-2.03 (m, 2H), 2.11-2.14 (m, 2H), 5.70 (m, 1H), 7.78 (m, 4H), 8.17-8.24 (m, 1H), 8.28 (s, 1H), 8.53 (s, 1H), 9.88 (s, 1H) |
| 6 | 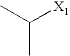 | | 310 | 423 | 1.17 (d, 6H), 1.56-1.69 (m, 2H), 1.80-1.91 (m, 2H), 1.93-2.05 (m, 2H), 2.11-2.23 (m, 2H), 3.49 (s, 3H), 4.04-4.14 (m, 1H), 5.64-5.77 (m, 1H), 7.75-7.83 (m, 4H), 7.80 (d, 1H), 8.53 (s, 1H), 9.86 (s, 1H) |
| 7 | 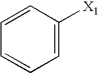 | | 305 | 457 | 1.54-1.66 (m, 2H), 1.78-1.90 (m, 2H), 1.92-2.02 (m, 2H), 2.12-2.25 (m, 2H), 3.48 (s, 3H), 5.69 (m, 1H), 7.49-7.61 (m, 3H).7.63-7.74 (m, 4H), 7.96 (d, 2H), 8.48 (s, 1H), 9.59 (s, 1H), 10.14 (s, 1H) |
| 8 | 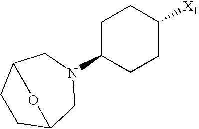 | | 310 | 574 | 1.18-1.42 (m, 4H), 1.57-1.69 (m, 4H), 1.75-1.92 (m, 8H), 1.93-2.03 (m, 1H), 2.07-2.24 (m, 3H), 3.49 (s, 3H), 3.64-3.75 (m, 1H), 4.20 (s, 2H), 5.70 (m 1H), 7.74-7.82 (m, 4H), 7.95 (s, 1H), 8.53 (s, 1H), 9.85 (s, 1H) |
| 9 | 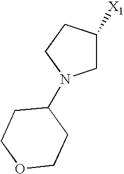 | | 314 | 534 | 1.56-2.08 (m, 10H), 2.04-2.26 (m, 2H), 3.07-3.19 (m, 2H), 3.50 (s, 3H), 3.54-3.67 (m, 2H), 3.90-4.00 (m, 2H), 4.48-4.77 (m, 1H), 5.71 (m, 1H), 7.78-7.98 (m, 4H), 8.60-8.75 (m, 2H), 9.92 (s, 1H) |
| 10 | 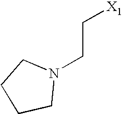 | | 310 | 478 | 1.56-1.67 (m, 2H), 1.64-1.78 (m, 4H), 1.80-1.92 (m, 2H), 2.12-2.24 (m, 2H), 2.62-2.72 (m, 4H), 2.72-2.79 (m, 2H), 3.49 (s, 3H), 5.64-5.75 (m, 1H), 7.75-7.84 (m, 4H), 8.28-8.34 (m, 1H), 8.53 (s, 1H), 9.88 (s, 1H) |

### EXAMPLE 11

### 4-(8-cyclopentyl-5-methyl-6,7-dioxo-5,6,7,8-tetrahydro-pteridin-2-ylamino)-3-methoxy-N-(1-methyl-piperidin-4-yl)-benzamide

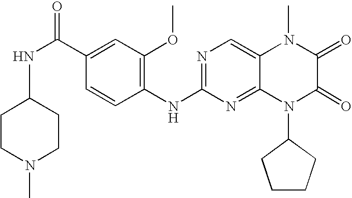

30 mg (0.11 mmol) 2-chloro-8-cyclopentyl-5-methyl-5,8-dihydropteridin-6,7-dione (method 1) are suspended in 0.3 ml isoamylalcohol and heated to 140° C. with 28 mg (0.11 mmol) 4-amino-3-methoxy-N-(1-methyl-piperidin-4-yl)-benzoic acid amide (J Pharm Sci. 1989, 78(10):829-32) and 25 mg (0.15 mmol) p-toluenesulphonic acid for 30 min. The reaction mixture is purified by column chromatography. The carrier material used is C18-RP-silica gel and a gradient is run through which consists of 95% water and 5% acetonitrile at the starting point and 2% water and 98% acetonitrile at the finishing point. 0.1% formic acid is added to both solvents. The compound is obtained as the formate.

Yield: 15 mg (0.030 mmol; 28%)

UV max: 322 nm

MS (ESI): 508 (M+H)⁺

¹H-NMR: 1.52-1.69 (m, 4H), 1.73-1.98 (m, 6H), 2.00-2.26 (m, 7H), 2.79-2.88 (m, 2H), 3.48 (s, 3H), 3.71-3.83 (m, 1H), 3.94 (s, 3H), 5.65 (m, 1H), 7.49-7.54 (m, 2H), 8.08-8.14 (m, 1H), 8.17-8.22 (m, 2H), 8.25 (s, 1H), 8.51 (s, 1H)

### EXAMPLES 12-13

The following compounds are by an analogous process to the one described in Example 11.

The aniline used to prepare the compounds is prepared by methods known from the literature (J Pharm Sci. 1989, 78(10):829-32; Bioorg Med Chem Lett. 2003 13(3):369-373 or J Med Chem. 1990, 33(11):3072-78).

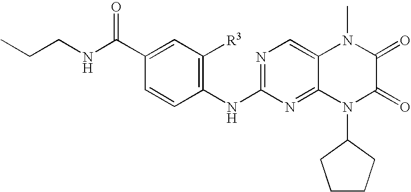

| | | UV | MS | |
|---|---|---|---|---|
| | | max | (ESI) | |
| # | R³ | [nm]: | (M + H)⁺: | NMR: |
| 12 | OCH₃ | 318 | 453 | 0.92 (t, 3H), 1.47-1.68 (m, 4H), 1.75-2.00 (m, 4H), 2.07-2.24 (m, 2H), 3.18-3.27 (m, 2H), 3.49 (s, 3H), 3.95 (s, 3H), 5.64 (m, 1H), 7.50-7.55 (m, 2H), 8.16-8.20 (m, 1H), 8.25 (m, 1H), 8.32-8.37 (m, 1H), 8.51 (s, 1H) |
| 13 | F | 306 | 441 | 0.90 (t, 3H), 1.48-1.66 (m, 4H), 1.72-1.88 (m, 4H), 2.05-2.21 (m, 2H), 3.18-3.24 (m, 2H), 3.48 (s, 3H), 5.59 (m, 1H), 7.68-7.76 (m, 2H), 7.88-7.96 (m, 1H), 8.42 (s, 1H), 8.48 (s, 1H), 9.32 (s, 1H) |

### EXAMPLE 14

### 4-(8-cyclopentyl-6-methyl-7-oxo-7,8-dihydro-pteridin-2-ylamino)-3-methoxy-N-(1-methyl-piperidin-4-yl)-benzamide

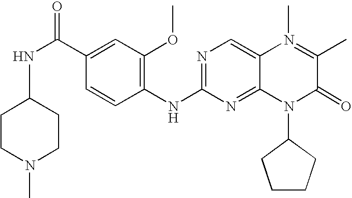

70 mg (0.177 mmol) of 4-(8-cyclopentyl-6-methyl-7-oxo-7.8-dihydro-pteridin-2-ylamino)-3-methoxy-benzoic acid (method 3), 1.3 ml (10 mmol) triethylamine, 74 mg (0.230 mmol) O-(benzotriazol-1-yl)-N,N,N′,N′-tetramethyluronium-tetrafluoroborate and 20 mg (0.177 mmol) 1-methyl-piperidine are dissolved in 3 ml dichloromethane.

After 5 h at 25° C. the solvent is eliminated in vacuo. The crude product is purified by column chromatography. The carrier material used is C18-RP-silica gel and a gradient is run through which consists of 95% water and 5% acetonitrile at the starting point and 2% water and 98% acetonitrile at the finishing point. 0.1% formic acid is added to both solvents. The compound is obtained as the formate.

Yield: 12 mg (mmol; 14%)

UV max: 363 nm

MS (ESI): 492 (M+H)⁺

¹H-NMR: 1.53-1.68 (m, 4H), 1.74-1.91 (m, 6H), 1.96-2.04 (m, 2H), 2.14-2.25 (m, 5H), 2.36 (s, 3H), 2.78-2.85 (m, 2H), 3.91 (s, 3H), 5.66 (m, 1H), 7.51-7.56 (m, 2H), 8.04 (d, 1H), 8.17 (d, 1H), 8.26 (s, 1H), 8.76 (s, 1H), 8.77 (s, 1H)

### EXAMPLE 15

The following compound is prepared analogously to Example 14.

The amine used to prepare the amide is commercially obtainable or may be prepared by the process described in method 2.

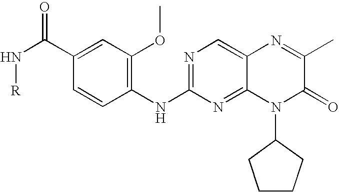

| | | | UV | MS | |
|---|---|---|---|---|---|
| | | | max | (ESI) | |
| # | R—X₁: | salt: | [nm]: | (M + H)⁺: | NMR: |
| 15 | 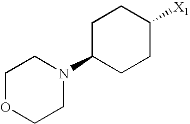 | HCOOH | 363 | 562 | 1.22-1.44 (m, 4H), 1.53+144 1.62 (m, 2H), 1.75-1.95 (m, 8H), 2.15-2.26 (m, 3H), 2.36 (s, 3H), 3.53-3.59 (m, 4H), 3.69-3.78 (m, 1H), 3.91 (s, 3H), 5.65 (m, 1H), 7.50-7.55 (m, 2H), 8.03 (d, 1H), 8.13 (d, 1H), 8.34 (s, 1H), 8.74-8.80 (m, 2H) |

### EXAMPLE 16

### 4-(8-cyclopentyl-5-methyl-7-oxo-7,8-dihydro-pyrido[2,3-d]pyrimidin-2-ylamino)-N-propyl-benzamide

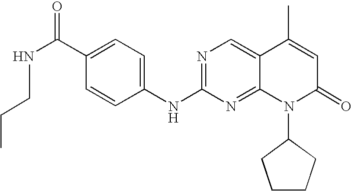

27 mg (0.088 mmol) 8-cyclopentyl-2-methanesulphonyl-5-methyl-8H-pyrido[2,3-d]pyrimidin-7-one is dissolved in 0.5 ml 2-butanol and combined with 23 mg (0.1 mmol) 4-aminobenzoic acid-N-propylamide hydrochloride.

After 15 h at 100° C. the solvent is eliminated in vacuo. The crude product is purified by column chromatography. The carrier material used is C18-RP-silica gel and a gradient is run through which consists of 95% water and 5% acetonitrile at the starting point and 5% water and 95% acetonitrile at the finishing point. 0.1% formic acid is added to both solvents.

Yield: 11 mg (mmol; 31%)

UV max: 350 nm

MS (ESI): 406 (M+H)⁺

¹H-NMR: 0.83-0.94 (m, 3H), 1.47-1.69 (m, 4H), 1.73-1.86 (m, 2H), 1.89-2.03 (m, 2H), 2.19-2.32 (m, 2H), 2.39 (s, 3H), 3.16-3.26 (m, 2H), 5.81-5.93 (m, 1H), 6.21-6.26 (m, 1H), 7.77-7.87 (m, 4H), 8.26-8.35 (m, 1H), 8.87 (s, 1H), 10.18 (s, 1H)

### EXAMPLES 17-25

The following compounds are prepared by an analogous process to the one described in Example 14. The preparation of the benzoic acid derivatives is described in method 5. The amine used to prepare the amide is commercially obtainable.

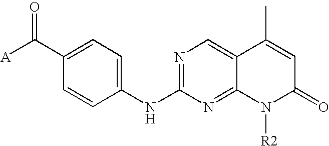

| | | | UV | MS | |
|---|---|---|---|---|---|
| | | | max | (ESI) | |
| # | R2 | A | [nm]: | (M + H)⁺ | salt form/type of NMR equipment/NMR data |
| 17 | 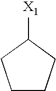 | 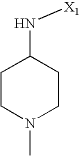 | 306, 350 | 461 | dihydrochloride/500 MHz/1.58-1.69 (m, 2H), 1.75-1.85 (m, 2H), 1.87-2.04 (m, 6H), 2.20-2.31 (m, 2H), 2.37-2.42 (s, 3H), 2.69-2.79 (m, 3H), 3.02-3.14 (m, 2H), 3.23-3.33 (m, 1H), 3.37-3.47 (m, 2H), 3.96-4.20 (m, 1H), 5.83-5.94 (m, 1H), 6.25 (s, 1H), 7.80-7.98 (m, 4H), 8.20-8.43 (m, 1H), 8.88 (s, 1H), 10.13-10.25 (m, 1H), 10.36-10.55 (m, 1H) |
| 18 | 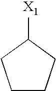 | 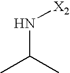 | 304, 348 | 406 | hydrochloride/500 MHz/1.14-1.21 (m, 6H), 1.57-1.69 (m, 2H), 1.74-1.85 (m, 2H), 1.91-2.03 (m, 2H), 2.20-2.31 (m, 2H), 2.34 (s, 3H), 4.05-4.15 (m, 1H), 5.83-5.93 (m, 1H), 6.24 (s, 1H), 7.78-7.87 (m, 4H), 8.02-8.09 (m, 1H), 8.87 (s, 1H), 10.18 (s, 1H) |
| 19 | 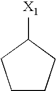 | 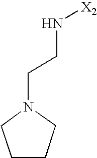 | 305, 350 | 461 | dihydrochloride/500 MHz/1.58-1.71 (m, 2H), 1.74-2.07 (m, 8H), 2.20-2.32 (m, 2H), 2.34-2.44 (m, 3H), 2.98-3.10 (m, 2H), 3.28-3.39 (m, 2H), 3.57-3.70 (m, 4H), 5.82-5.94 (m, 1H), 6.25 (s, 1H), 7.82-7.99 (m, 4H), 8.71-8.80 (m, 1H), 8.88 (s, 1H), 10.26 (s, 1H), 10.41-10.56 (m, 1H) |
| 20 | 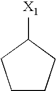 | 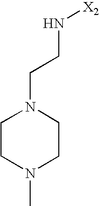 | 306, 350 | 490 | trihydrochloride/500 MHz/1.58-1.68 (m, 2H), 1.74-1.86 (m, 2H), 1.91-2.04 (m, 2H), (s, 3H), 2.21-2.49 (m, 15H), 5.82-5.93 (m, 1H), 6.24 (s, 1H), 7.78-7.86 (m, 4H), 8.21-8.27 (m, 1H), 8.87 (s, 1H), 10.21 (s, 1H) |
| 21 | 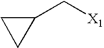 | 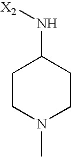 | 305, 350 | 447 | dihydrochloride/500 MHz/0.39-0.47 (m, 4H), 1.32-1.42 (m, 1H), 1.84-2.05 (m, 4H), 2.43 (s, 3H), 2.69-2.79 (m, 3H), 3.02-3.14 (m, 2H), 3.25-3.32 (m, 1H), 3.38-3.47 (m, 2H), 3.96-4.06 (m, 1H), 4,18-4.25 (m, 2H), 6.32 (s, 1H), 7.85-7.95 (m, 4H), 8.17-8.41 (m, 1H), 8.90 (s, 1H), 10.32 (s, 2H) |
| 22 | 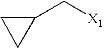 | 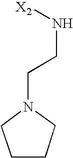 | 305, 350 | 447 | dihydrochloride/500 MHz/0.41-0.47 (m, 4H), 1.32-1.41 (m, 1H), 1.83-2.07 (m, 4H), 2.43 (s, 3H), 2.97-3.09 (m, 2H), 3.28-3.37 (m, 2H), 3.57-3.68 (m, 4H), 4.17-4.24 (m, 2H), 6.32 (s, 1H), 7.89-7.98 (m, 4H), 8.73-8.82 (m, 1H), 8.90 (s, 1H), 10.35 (s, 1H), 10.51 (s, 1H) |
| 23 | 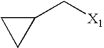 | 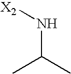 | 305, 350 | 392 | hydrochloride/500 MHz/0.39-0.47 (m, 4H), 1.10-1.21 (m, 7H), 1.31-1.42 (m, 1H), 2.43 (s, 3H), 4.05-4.15 (m, 1H), 4.17-4.25 (m, 2H), 6.31 (s, 1H), 7.81-8.13 (m, 5H), 8.89 (s, 1H), 10.28 (s, 1H) |
| 24 | 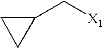 | 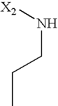 | 305, 350 | 392 | hydrochloride/500 MHz/0.40-0.47 (m, 4H), 0.86-0.93 (m, 3H), 1.31-1.42 (m, 1H), 1.49-1.59 (m, 2H), 2.42 (s, 3H), 3.17-3.26 (m, 2H), 4.17-4.24 (m, 2H), 6.31 (s, 1H), 7.81-7.92 (m, 4H), 8.28-8.36 (m, 1H), 8.89 (s, 1H), 10.29 (s, 1H) |
| 25 | 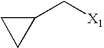 | 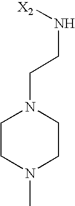 | 305, 350 | 476 | trihydrochioride/500 MHz/0.41-0.48 (m, 4H), 1.32-1.41 (m, 1H), 2.43 (s, 3H), 2.84 (s, 3H), 4.17-4.24 (m, 2H), 6.32 (s, 1H), 7.88-7.98 (m, 4H), 8.71-8.80 (m, 1H), 8.90 (s, 1H), 10.36 (s, 1H), |

### EXAMPLES 26-40

The following compounds are prepared by an analogous process to the one described in Example 14. The preparation of the benzoic acid derivative is described in method 5. The amine used to prepare the amide is commercially obtainable or described in method 6.

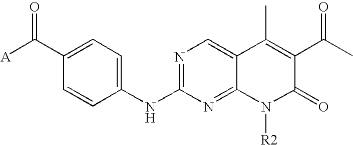

| | | | UV | MS | |
|---|---|---|---|---|---|
| | | | max | (ESI) | |
| # | A | R₃′ | [nm]: | (M + H)⁺: | salt form/type of NMR equipment/NMR data: |
| 26 | 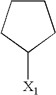 | 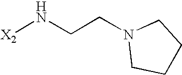 | 306.4 | 503 | dihydrochloride/500 MHz/1.56-2.05 (m, 9H), 2.19-2.34 (m, 5H), 2.43 (s, 3H), 2.84-3.01 (m, 7H), 5.81-5.96 (m, 1H), 7.77-7.93 (m, 4H), 8.45-8.56 (m, 1H), 9.02 (s, 1H), 10.36 (s, 1H) |
| 27 | 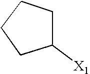 | 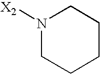 | 306, 358 | 474 | hydrochloride/400 MHz/1.40-1.68 (m, 8H), 1.72-1.99 (m, 4H), 2.18-2.36 (m, 5H), 2.43 (s, 3H), 3.20-3.66 (m, 4H), 5.80-5.93 (m, 1H), 7.32-7.42 (m, 2H), 7.73-7.82 (m, 2H), 9.00 (s, 1H), 10.27 (s, 1H) |
| 28 | 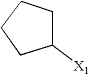 | 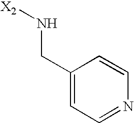 | 306, 358 | 497 | dihydrochloride/400 MHz/1.56-1.72 (m, 2H), 1.76-1.90 (m, 2H), 1.92-2.06 (m, 2H), 2.19-2.37 (m, 5H), 2.43 (s, 3H), 4.68-4.79 (m, 2H), 5.85-5.97 (m, 1H), 7.85-8.04 (m, 6H), 8.82-8.90 (m, 2H), 9.04 (s, 1H), 9.31-9.40 (m, 1H), 10.43 (s, 1H) |
| 29 | 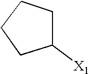 | 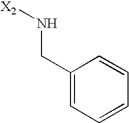 | 306, 358 | 496 | hydrochloride/400 MHz/1.56-1.71 (m, 2H), 1.75-2.04 (m, 4H), 2.19-2.37 (m, 5H), 2.43 (s, 3H), 4.43-4.55 (m, 2H), 5.83-5.97 (m, 1H), 7.19-7.37 (m, 5H), 7.79-7.96 (m, 4H), 8.86-8.96 (m, 1H), 9.02 (s, 1H), 10.36 (s, 1H) |
| 30 | 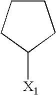 | 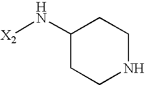 | 306, 358 | 489 | dihydrochloride/400 MHz/1.56-1.71 (m, 2H), 1.72-1.90 (m, 4H), 1.91-2.04 (m, 4H), 2.19-2.37 (m, 5H), 2.43 (s, 3H), 2.93-3.08 (m, 2H), 3.25-3.37 (m 2H), 4.00-4.12 (m, 1H), 5.84-5.97 (m, 1H), 7.79-7.93 (m, 4H), 8.30-8.39 (m, 1H), 8.64-8.86 (m, 2H), 9.02 (s, 1H), 10.35 (s, 1H) |
| 31 | 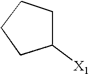 | 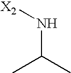 | 306, 358 | 448 | hydrochloride/400 MHz/1.12-1.22 (m, 6H), 1.56-1.71 (m, 2H), 1.76-1.90 (m, 2H), 1.91-2.05 (m, 2H), 2.19-2.37 (m, 5H), 2.43 (s, 3H), 4.04-4.19 (m, 1H), 5.84-5.98 (m, 1H), 7.75-7.90 (m, 4H), 8.00-8.11 (m, 1H), 9.02 (s, 1H), 10.32 (s, 1H) |
| 32 | 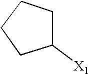 | 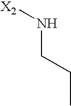 | 306, 358 | 448 | hydrochloride/400 MHz/0.84-0.95 (m, 3H), 1.46-1.72 (m, 4H), 1.76-1.90 (m, 2H), 1.90-2.04 (m, 2H), 2.19-2.36 (m, 5H), 2.43 (s, 3H), 3.16-3.27 (m, 2H), 5.83-5.97 (m, 1H), 7.76-7.90 (m, 4H), 8.25-8.36 (m, 1H), 9.02 (s, 1H), 10.32 (s, 1H) |
| 33 | 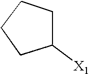 | 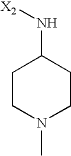 | 306, 358 | 503 | dihydrochloride/400 MHz/1.56.1-72 (m, 2H), 1.75-2.07 (m, 6H), 2.18-2.37 (m, 5H), (s, 3H), 2.64-2.77 (m, 3H), 3.00-3.14 (m, 2H), 3.36-3.47 (m, 2H), 3.95-4.08 (m, 1H), 5.85-5.97 (m, 1H), 7.79-7.97 (m, 4H), 8.38-8.47 (m, 1H), 9.02 (s, 1H), 10.36 (s, 1H), 10.61 (s, 1H) |
| 34 | 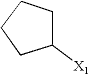 | 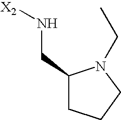 | 306, 358 | 517 | dihydrochioride/400 MHz/1.22-1.35 (m, 3H), 1.57-1.72 (m, 2H), 1.73-2.37 (mn, 13H), 2.43 (s, 3H), 3.01-3.16 (m, 2H), 3.33-3.82 (m, 5H), 5.83-5.97 (m, 1H), 7.82-8.01 (m, 4H), 8.92-9.00 (m, 1H), 9.03 (s, 1H), 10.35-10.50 (m, 2H) |
| 35 | 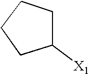 | 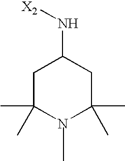 | 306, 358 | 559 | dihydrochloride/400 MHz/1.40 (s, 6H), 1.52 (s, 6H), 1.57-1.70 (m, 2H), 1.77-1.89 (m, 2H), 1.90-2.13 (m, 6H), 2.19-2.36 (m, 5H), 2.43 (s, 3H), 2.64-2.72 (m, 3H), 4.28-4.42 (m, 1H), 5.83-5.97 (m, 1H), 7.79-7.94 (m, 4H), 8.41-8.49 (m, 1H), 9.02 (s, 1H), 9.93-10.04 (m, 1H), 10.36 (s, 1H) |
| 36 | 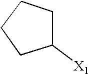 | 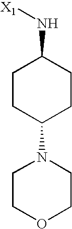 | 306, 358 | 573 | dihydrochloride/400 MHz/1.34-1.49 (m, 2H), 1.55-1.70 (m, 4H), 1.76-1.89 (m, 2H), 1.90-2.04 (m, 4H), 2.14-2.47 (m, 10H), 3.02-3.23 (m, 3H), 3.35-3.45 (m, 2H), 3.70-4.02 (m, 5H), 5.84-5.97 (m, 1H), 7.74-7.90 (m, 4H), 8.14-8.21 (m, 1H), 9.02 (s, 1H), 10.34 (s, 1H), 10.97 (m, 1H) |
| 37 | 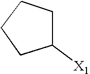 | 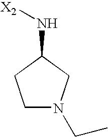 | 306, 358 | 503 | dihydrochioride/400 MHz/1.20-1.31 (m, 3H), 1.55-1.72 (m, 2H), 1.90-2.47 (m, 10H), 3.00-3.39 (m, 4H), 5.83-5.97 (m, 1H), 7.80-8.02 (in, 4H), 8.68-8.83 (in, IH), 9.03 (s, 1H), 10.38 (s, 1H), 10.65-11.05 (m, 1H) |
| 38 | 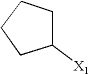 | 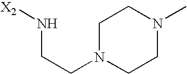 | 306, 358 | 532 | trihydrochloride/400 MHz/1.57-1.71 (m, 2H), 1.75-1.90 (m, 2H), 1.90-2.04 (m, 2H), 2.18-2.37 (m, 5H), 2.43 (s, 3H), 2.84 (s, 3H), 3.18-4.04 (m, 12H), 5.82-5.97 (m, 1H), 7.80-8.01 (m, 4H), 8.71-8.84 (m, 1H), 9.03 (s, 1H), 10.39 (s, 1H) |
| 39 | 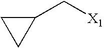 | 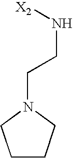 | 306, 358 | 489 | dihydrochloride/400 MHz/0.41-0.51 (m, 4H), 1.32-1.43 (m, 1H), 1.82-2.08 (m, 4H), (s, 3H), 2.46 (s, 3H), 2.98-3.12 (m, 2H), 3.28-3.38 (m, 2H), 3.57-3.70 (m, 4H), 4.21-4.27 (m, 2H), 7.88-7.98 (m, 4H), 8.67-8.76 (m, 1H), 9.04 (s, 1H), |
| 40 | 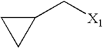 | 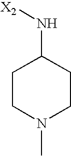 | 306, 356 | 489 | dihydrochloride/400 MHz/0.41-0.50 (m, 4H), 1.32-1.45 (m, 1H), 1.80-2.07 (m, 4H), 2.37 (s, 3H), 2.46 (s, 3H), 2.65.2.80 (m, 3H), 3.01-3.49 (m, 4H), 3.95-4.09 (m, 1H), 4.21-4.28 (m, 2H), 7.86-7.94 (m, 4H), 8.36-8.43 (m, 1H), 9.04 (s, 1H), |

Biological Properties

As demonstrated by DNA staining followed by FACS analysis, the inhibition of proliferation brought about by the compounds according to the invention is mediated above all by the arrest of the cells in the G2/M phase of the cell cycle. The cells arrest, depending on the type of cell used, for a specific length of time in this cell cycle phase before programmed cell death is initiated. An arrest in the G2/M phase of the cell cycle may be initiated e.g. by the inhibition of specific cell cycle kinases. On the basis of their biological properties the compounds of general formula I according to the invention, their isomers and the physiologically acceptable salts thereof are suitable for treating diseases characterised by excessive or anomalous cell proliferation.

Such diseases include for example: viral infections (e.g. HIV and Kaposi's sarcoma); inflammatory and autoimmune diseases (e.g. colitis, arthritis, Alzheimer's disease, glomerulonephritis and wound healing); bacterial, fungal and/or parasitic infections; leukaemias, lymphomas and solid tumours; skin diseases (e.g. psoriasis); bone diseases; cardiovascular diseases (e.g. restenosis and hypertrophy). They are also useful for protecting proliferating cells (e.g. hair, intestinal, blood and progenitor cells) from DNA damage caused by radiation, UV treatment and/or cytostatic treatment (Davis et al., 2001). The new compounds may be used for the prevention, short- or long-term treatment of the above-mentioned diseases, also in combination with other active substances used for the same indications, e.g. cytostatics, steroids or antibodies.

Example PLK-1 Kinase Assay

Recombinant human PLK1 enzyme linked to GST at its N-terminal end is isolated from insect cells infected with baculovirus (Sf21). Purification is carried out by affinity chromatography on glutathione sepharose columns.

4×10⁷ Sf21 cells (Spodoptera frugiperda) in 200 ml of Sf-900 II Serum free insect cell medium (Life Technologies) are seeded in a spinner flask. After 72 hours' incubation at 27° C. and 70 rpm, 1×10⁸ Sf21 cells are seeded in a total of 180 ml medium in a new spinner flask. After another 24 hours, 20 ml of recombinant Baculovirus stock suspension are added and the cells are cultivated for 72 hours at 27° C. at 70 rpm. 3 hours before harvesting, okadaic acid is added (Calbiochem, final concentration 0.1 μM) and the suspension is incubated further. The cell number is determined, the cells are removed by centrifuging (5 minutes, 4° C., 800 rpm) and washed 1× with PBS (8 g NaCl/l, 0.2 g KCl/l, 1.44 g Na₂HPO₄/l, 0.24 g KH₂PO4/1). After centrifuging again the pellet is flash-frozen in liquid nitrogen. Then the pellet is quickly thawed and resuspended in ice-cold lysing buffer (50 mM HEPES pH 7.5, 10 mM MgCl₂, 1 mM DTT, 5 μg/ml leupeptin, 5 μg/ml aprotinin, 100 μM NaF, 100 μM PMSF, 10 mM β-glycerolphosphate, 0.1 mM Na₃VO₄, 30 mM 4-nitrophenylphosphate) to give 1×10⁸ cells/17.5 ml. The cells are lysed for 30 minutes on ice. After removal of the cell debris by centrifugation (4000 rpm, 5 minutes) the clear supernatant is combined with glutathione sepharose beads (1 ml resuspended and washed beads per 50 ml of supernatant) and the mixture is incubated for 30 minutes at 4° C. on a rotating board. Then the beads are washed with lysing buffer and the recombinant protein is eluted from the beads with 1 ml eluting buffer/ml resuspended beads (eluting buffer: 100 mM Tris/HCl pH=8.0, 120 mM NaCl, 20 mM reduced glutathione (Sigma G-4251), 10 mM MgCl₂, 1 mM DTT). The protein concentration is determined by Bradford Assay.

Assay

The following components are combined in a well of a 96-well round-bottomed dish (Greiner bio-one, PS Microtitre plate No. 650101):
10 μl of the compound to be tested in variable concentrations (e.g. beginning at 300 μM, and dilution to 1:3) in 6% DMSO, 0.5 mg/ml casein (Sigma C-5890), 60 mM β-glycerophosphate, 25 mM MOPS pH=7.0, 5 mM EGTA, 15 mM MgCl₂, 1 mM DTT
20 μl substrate solution (25 mM MOPS pH=7.0, 15 mM MgCl₂, 1 mM DTT, 2.5 mM EGTA, 30 mM β-glycerophosphate, 0.25 mg/ml casein)
20 μl enzyme dilution (1:100 dilution of the enzyme stock in 25 mM MOPS pH=7.0, 15 mM MgCl₂, 1 mM DTT)
10 μl ATP solution (45 μM ATP with 1.11×10⁶ Bq/ml gamma-P33-ATP).

The reaction is started by adding the ATP solution and continued for 45 minutes at 30° C. with gentle shaking (650 rpm on an IKA Schüttler MTS2). The reaction is stopped by the addition of 125 μl of ice-cold 5% TCA per well and incubated on ice for at least 30 minutes. The precipitate is transferred by harvesting onto filter plates (96-well microtitre filter plate: UniFilter-96, GF/B; Packard; No. 6005177), then washed four times with 1% TCA and dried at 60° C. After the addition of 35 μl scintillation solution (Ready-Safe; Beckmann) per well the plate is sealed shut with sealing tape and the amount of P33 precipitated is measured with the Wallac Betacounter. The measured data are evaluated using the standard Graphpad software (Levenburg-Marquard Algorhythmus).

The activity of the compounds according to the invention is determined in the cytotoxicity test on cultivated human tumour cells and/or in a FACS analysis, for example on HeLa S3 cells. In both test methods the compounds exhibit good to very good activity, i.e. for example an EC50 value in the HeLa S3 cytotoxicity test of less than 5 μmol/L, generally less than 1 μmol/L.

Measurement of Cytotoxicity on Cultivated Human Tumour Cells

To measure cytotoxicity on cultivated human tumour cells, cells of cervical carcinoma tumour cell line HeLa S3 (obtained from American Type Culture Collection (ATCC)) are cultivated in Ham's F12 Medium (Life Technologies) and 10% foetal calf serum (Life Technologies) and harvested in the log growth phase. Then the HeLa S3 cells are placed in 96-well plates (Costar) at a density of 1000 cells per well and incubated overnight in an incubator (at 37° C. and 5% CO₂), while on each plate 6 wells are filled with medium alone (3 wells as the medium control, 3 wells for incubation with reduced AlamarBlue reagent). The active substances are added to the cells in various concentrations (dissolved in DMSO; DMSO final concentration: 0.1%) (in each case as a triple measurement). After 72 hours incubation 20 μl AlamarBlue reagent (AccuMed International) are added to each well, and the cells are incubated for a further 5-7 hours. As a control, 20 μl reduced AlamarBlue reagent is added to each of 3 wells (AlamarBlue reagent, which is autoclaved for 30 min). After incubation the colour change of the AlamarBlue reagent in the individual wells is determined in a Perkin Elmer fluorescence spectrophotometer (excitation 530 nm, emission 590 nm, slits 15, integrate time 0.1). The amount of AlamarBlue reagent reacted represents the metabolic activity of the cells. The relative cell activity is calculated as a percentage of the control (HeLa S3 cells without inhibitor) and the active substance concentration which inhibits the cell activity by 50% (IC50) is derived. The values are calculated from the average of three individual measurements—with correction of the dummy value (medium control).

FACS Analysis

Propidium iodide (PI) binds stoichiometrically to double-stranded DNA, and is thus suitable for determining the proportion of cells in the G1, S, and G2/M phase of the cell cycle on the basis of the cellular DNA content. Cells in the G0 and G1 phase have a diploid DNA content (2N), whereas cells in the G2 or mitosis phase have a 4N DNA content.

For PI staining, for example, 1×10⁶ HeLa S3 cells are seeded onto a 75 cm2 cell culture flask, and after 24 h either 0.1% DMSO is added as control or the substance is added in various concentrations (in 0.1% DMSO). The cells are incubated for 24 h with the substance or with DMSO before the cells are washed 2× with PBS and then detached with trypsin/EDTA. The cells are centrifuged (1000 rpm, 5 min, 4° C.), and the cell pellet is washed 2× with PBS before the cells are resuspended in 0.1 ml PBS. Then the cells are fixed with 80% ethanol for 16 hours at 4° C. or alternatively for 2 hours at −20° C. The fixed cells are centrifuged (1000 rpm, 5 min, 4° C.), washed with PBS and then centrifuged again. The cell pellet is resuspended in 2 ml 0.25% Triton X-100 in PBS, and incubated on ice for 5 min before 5 ml PBS are added and the mixture is centrifuged again. The cell pellet is resuspended in 350 μl PI staining solution (0.1 mg/ml RNase A (Sigma, No. R-4875), 10 μg/ml prodium iodide (Sigma, No. P-4864) in 1×PBS). The cells are incubated for 20 min in the dark with the staining buffer before being transferred into sample measuring containers for the FACS scan. The DNA measurement is carried out in a Becton Dickinson FACS Analyzer, with an argon laser (500 mW, emission 488 nm), and the DNA Cell Quest Programme (BD). The logarithmic PI fluorescence is determined with a band-pass filter (BP 585/42). The cell populations in the individual cell cycle phases are quantified using the ModFit LT Programme made by Becton Dickinson.

The compounds according to the invention are also tested accordingly for other tumour cells. For example, these compounds are effective on carcinomas of all kinds of tissue (e.g. breast (MCF7); colon (HCT116), head and neck (FaDu), liver (HepG2), lung (NCI-H460), stomach (NCI-N87), pancreas (BxPC-3), prostate (DU145)), sarcomas (e.g. SK-UT-1B, Saos-2), leukaemias and lymphomas (e.g. HL-60, THP-1, Raji, Jurkat, GRANTA-519) and other tumours (e.g. melanomas (BRO), gliomas (U-87MG)) and could be used for such indications. This is evidence of the broad applicability of the compounds according to the invention for the treatment of all kinds of tumour types.

The compounds of general formula (I) may be used on their own or in conjunction with other active substances according to the invention, optionally also in conjunction with other pharmacologically active substances.

Suitable preparations include for example tablets, capsules, suppositories, solutions, particularly solutions for injection (s.c., i.v., i.m.) and infusion, elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation examples which follow illustrate the present invention without restricting its scope:

Examples of Pharmaceutical Formulations

A)

| Tablets | per tablet | |
|---|---|---|
| active substance | 100 | mg |
| lactose | 140 | mg |
| corn starch | 240 | mg |
| polyvinylpyrrolidone | 15 | mg |
| magnesium stearate | 5 | mg |
| | 500 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

B)

| Tablets | per tablet | |
|---|---|---|
| active substance | 80 | mg |
| lactose | 55 | mg |
| corn starch | 190 | mg |
| microcrystalline cellulose | 35 | mg |
| polyvinylpyrrolidone | 15 | mg |
| sodium-carboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

C)

Ampoule solution

| | | |
|---|---|---|
| active substance | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

What is clamed:

1) A Compound of the formula (1) 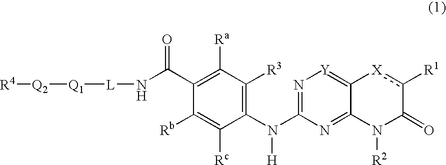
wherein
the dotted line denotes an optional bond, while
X denotes N or C—R^{e}, if X and CR¹ are linked by a double bond, or
X denotes —N—R^{d}, if X—CR¹ is linked by a single bond,
Y denotes N or CH,
R¹ denotes a group selected from among hydrogen, halogen, ═O, —OR⁵, —C(═O)R⁶, —C(═O)NR⁵R⁶, —NR⁵R⁶, —NR⁵C(═O)R⁶, —NR⁵SO₂R⁶, —N═CR⁵R⁶, —SR⁵, —SOR⁵, —SO₂R⁵, —SO₂NR⁵R⁶ and pseudohalogen,
or an optionally mono- or polysubstituted group selected from among C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen, or if X and CR¹ are linked by a single bond, the group ═O as well;
R² denotes hydrogen or an optionally mono- or polysubstituted group selected from among C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
R³ denotes a group selected from among hydrogen, halogen, —OR⁵, —C(═O)R⁵, —C(═O)NR⁵R⁶, —NR⁵R⁶, —NR⁵C(═O)R⁶, —NR⁵SO₂R⁶, —N═CR⁵R⁶, —SR⁵, —SOR⁵, —SO₂R⁵, —SO₂NR⁵R⁶ and pseudohalogen,
or an optionally mono- or polysubstituted group selected from among C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen:
L denotes a bond or a group selected from among optionally mono- or polysubstituted C₁₋₁₆-alkyl, C₂₋₁₆-alkenyl and C₂₋₁₆-alkynyl, while the substituent(s) may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
Q₁ and Q₂ each independently of one another denote a bond or a group selected from among optionally mono- or polysubstituted C₁₋₁₆alkyl, C₂₋₁₆-alkenyl, C₂₋₁₆-alkynyl, C₃₋₁₀cycloalkyl, aryl, heterocyclyl and heteroaryl, while the substituent(s) may be identical or different and are selected from among halogen, —NO₂, R⁵, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR₅SO₂NR⁶R⁷, —OSO₂NR⁵R⁶ and pseudohalogen;
R⁴ denotes hydrogen or a group selected from among optionally mono- or polysubstituted C₁₋₆alkyl, C₂₋₁₆alkenyl, C₂₋₁₆alkynyl, C₃₋₁₀cycloalkyl, aryl, heterocyclyl and heteroaryl, while the substituent(s) may be identical or different and are selected from among —NO₂, R⁵, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
R^{a}, R^{b}, R^{c}, each independently of one another denote a group selected from among hydrogen, halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen; or
an optionally mono- or polysubstituted group selected from among C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen and pseudohalogen;
R^{d} denotes hydrogen or a group selected from among optionally mono- or polysubstituted C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵ SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
R^{e} denotes a group selected from among hydrogen, halogen, pseudohalogen or a group selected from among optionally mono- or polysubstituted C₁₋₆alkyl, C₂₋₆alkenyl, C₂₋₆alkynyl, C₃₋₆cycloalkyl, aryl, heterocyclyl and heteroaryl, while the substituents may be identical or different and are selected from among halogen, —NO₂, —OR⁵, —C(═O)R⁵, —C(═O)OR⁵, —C(═O)NR⁵R⁶, —NR⁵R⁶, —NR⁵C(═O)R⁶, —NR⁵C(═O)OR⁶, —NR⁵C(═O)NR⁶R⁷, —NR⁵SO₂R⁶, —N═CR⁵R⁶, —SR⁵, —SOR⁵, —SO₂R⁵, —SO₂NR⁵R⁶, —NR⁵SO₂NR⁶R⁷, —OSO₂NR⁵R⁶ and pseudohalogen;
R⁵, R⁶ and R⁷ each independently of one another denote hydrogen or a group selected from among optionally mono- or polysubstituted C₁₋₅alkyl, C₂₋₅alkenyl, C₂₋₅alkynyl, C₃₋₁₀cycloalkyl, aryl, heterocyclyl and heteroaryl, while the substituents may be identical or different and are selected from among C₃₋₁₀cycloalkyl, aryl, heterocyclyl, heteroaryl, halogen, —NO₂, —OR⁸, —C(═O)R⁸, —C(═O)OR⁸, —C(═O)NR⁸R⁹, —NR⁸R⁹, —NR⁸C(═O)R⁹, —NR⁸C(═O)OR⁹, —NR⁸C(═O)NR⁹R¹⁰, —NR⁸C(═O)ONR⁹R¹⁰, —NR⁸SO₂R⁹, —N═CR⁸R⁹, —SR⁸, —SOR⁸, —SO₂R⁸, —SO₂NR⁸R⁹, —NR⁸SO₂NR⁹R¹⁰, —OSO₂NR⁸R⁹ and pseudohalogen;
R⁸, R⁹ and R¹⁰ each independently of one another denote hydrogen or a group selected from among optionally substituted C₁₋₈alkyl, C₂₋₈alkenyl, C₂₋₈alkynyl, C₃₋₁₀cycloalkyl, aryl, heterocyclyl and heteroaryl, while the substituent(s) may be identical or different and are selected from among halogen, —NH₂, —OH and pseudohalogen;
optionally in the form of the tautomers, racemates, enantiomers, diastereomers and mixtures thereof, and optionally the pharmacologically acceptable acid addition salts thereof.

2) The Compound according to claim 1, wherein
Y denotes CH.

3) The Compound according to claim 2, wherein
R^{c} denotes a group selected from among hydrogen, —F, —Cl, methyl and ethyl.

4) The Compound according to claim 3, wherein
R^{a} and R^{b} each independently of one another denote hydrogen or fluorine;
or an optionally mono- or polysubstituted group selected from among C₁₋₂, C₂alkenyl, C₂alkynyl, C₃₋₆cycloalkyl, aryl, heterocyclyl and heteroaryl, while the substituents may be identical or different and are selected from among hydrogen, halogen, —NO₂, —OR⁴, —C(═O)R⁴, —C(═O)OR⁴, —C(═O)NR⁴R⁵, —NR⁴R⁵, —NR⁴C(═O)R⁵, —NR⁴C(═O)OR⁵, —NR⁴C(═O)NR⁵R⁶, —NR⁴SO₂R⁵, —N═CR⁴R⁵, —SR⁴, —SOR⁵, —SO₂R⁴, —SO₂NR⁴R⁵, —NR⁴, —SO₂NR⁴R⁵, —OSO₂NR⁴R⁵ and pseudohalogen.

5) The Compound according to claim 4, wherein
R^{a} and R^{b} each independently of one another denote hydrogen or fluorine.

6) The Compound according to claim 5, wherein
R² denotes isopropyl or cyclopentyl.

7) A method of treating a disease chosen from cancer, bacterial and viral infections, inflammatory and autoimmune diseases, chemotherapy-induced alopecia and mucositis, cardiovascular diseases, nephrological diseases and chronic and acute neurodegenerative diseases comprising administering to a patient a therapeutically effective amount of a compound of formula (1) according to one of claim 1.

8) A Pharmaceutical composition comprising a pharmaceutically effective amount compound of the formula (I) according to claim 1 and optionally pharmaceutically acceptable excipients and/or carriers.
